SEQUENCE LISTING (28 sequences)

SEQ ID NO: 1 - protein, 230 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 2 - DNA, 1076 bp, Homo sapiens
  source 1..1076: mol_type = unassigned DNA
gggcagcgcctgacatgcctgatcctctcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggataaaaaaaaaaaaaaaaaaaaaa

SEQ ID NO: 3 - protein, 219 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRAERHVKEDDGRKSPGEVPPGTSPACILATWPPGLLVLLWQETTLPEHCFSWTLEAGTG

SEQ ID NO: 4 - protein, 222 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRPSQGSHLPSCLSKEPLGRRNPLPTHFHPSPPGLHLSHQDSSSQRPLGCSLAWTEARDTSTQ

SEQ ID NO: 5 - protein, 256 aa, Macaca fascicularis
MPDPLFSAVQGKDKILHKALCICPWPGKGGMEPLRLLILLFATELSGAHNTTVFQGVEGQSLQVSCPYDSMKHWGRRKAWCRQLGEKGPCQRVVSTHNLWLLSFLRRRNGSTAITDDTLGGTLTITLRNLQPHDAGFYQCQSLHGSEADTLRKVLVEVLADPLDHRDAGDLWVPGESESFEDAHVEHSISRSLLEGEIPEPPTSVLLLLACIFLIKILAASALWAAAWHGQKPGTHPPSEPDCGHDPGHQLQTLPG

SEQ ID NO: 6 - protein, 227 aa, Mus sp.
MGPLHQFLLLLITALSQALNTTVLQGMAGQSLRVSCTYDALKHWGRRKAWCRQLGEEGPCQRVVSTHGVWLLAFLKKRNGSTVIADDTLAGTVTITLKNLQAGDAGLYQCQSLRGREAEVLQKVLVEVLEDPLDDQDAGDLWVPEESSSFEGAQVEHSTSRNQETSFPPTSILLLLACVLLSKFLAASILWAVARGRQKPGTPVVRGLDCGQDAGHQLQILTGPGGT

SEQ ID NO: 7 - protein, 62 aa, Homo sapiens
LHGSEADTLRKVLVEVLADPLDHRDAGDLWFPGESESFEDAHVEHSISRSLLEGEIPFPPTS

SEQ ID NO: 8 - protein, 56 aa, Homo sapiens
LHGSEADTLRKVLVEVLADPLDHRDAGDLWFPGESESFEDAHVEHSISRAERHVKE

SEQ ID NO: 9 - protein, 59 aa, Homo sapiens
LHGSEADTLRKVLVEVLADPLDHRDAGDLWFPGESESFEDAHVEHSISRPSQGSHLPSC

SEQ ID NO: 10 - protein, 62 aa, Macaca fascicularis
LHGSEADTLRKVLVEVLADPLDHRDAGDLWVPGESESFEDAHVEHSISRSLLEGEIPFPPTS

SEQ ID NO: 11 - protein, 59 aa, Mus sp.
LRGREAEVLQKVLVEVLEDPLDDQDAGDLWVPEESSSFEGAQVEHSTSRNQETSFPPTS

SEQ ID NO: 12 - DNA, 50 bp, synthetic construct
  misc_feature 1..50: note = Description of Artificial Sequence: Synthetic oligonucleotide
  source 1..50: mol_type = other DNA
  modified_base 8: mod_base = OTHER; note = 5-[N-(1-naphthylmethyl)carboxamide]-2 prime-deoxyuridine (NapdU)
  modified_base 13: mod_base = OTHER; note = 5-[N-(1-naphthylmethyl)carboxamide]-2 prime-deoxyuridine (NapdU)
  modified_base 20: mod_base = OTHER; note = 5-[N-(1-naphthylmethyl)carboxamide]-2 prime-deoxyuridine (NapdU)
  modified_base 23: mod_base = OTHER; note = 5-[N-(1-naphthylmethyl)carboxamide]-2 prime-deoxyuridine (NapdU)
  modified_base 27: mod_base = OTHER; note = 5-[N-(1-naphthylmethyl)carboxamide]-2 prime-deoxyuridine (NapdU)
  modified_base 29..30: mod_base = OTHER; note = 5-[N-(1-naphthylmethyl)carboxamide]-2 prime-deoxyuridine (NapdU)
  modified_base 38: mod_base = OTHER; note = 5-[N-(1-naphthylmethyl)carboxamide]-2 prime-deoxyuridine (NapdU)
ccctcgatccggtggcggatcctaagtattggcccggtccgccaagaaga

SEQ ID NO: 13 - DNA, 50 bp, synthetic construct
  misc_feature 1..50: note = Description of Artificial Sequence: Synthetic oligonucleotide
  source 1..50: mol_type = other DNA
  modified_base 6: mod_base = OTHER; note = 5-[N-(1-naphthylmethyl)carboxamide]-2 prime-deoxyuridine (NapdU)
  modified_base 8: mod_base = OTHER; note = 5-[N-(1-naphthylmethyl)carboxamide]-2 prime-deoxyuridine (NapdU)
  modified_base 14: mod_base = OTHER; note = 5-[N-(1-naphthylmethyl)carboxamide]-2 prime-deoxyuridine (NapdU)
  modified_base 26..28: mod_base = OTHER; note = 5-[N-(1-naphthylmethyl)carboxamide]-2 prime-deoxyuridine (NapdU)
  modified_base 31: mod_base = OTHER; note = 5-[N-(1-naphthylmethyl)carboxamide]-2 prime-deoxyuridine (NapdU)
  modified_base 37: mod_base = OTHER; note = 5-[N-(1-naphthylmethyl)carboxamide]-2 prime-deoxyuridine (NapdU)
  modified_base 39: mod_base = OTHER; note = 5-[N-(1-naphthylmethyl)carboxamide]-2 prime-deoxyuridine (NapdU)
  modified_base 44: mod_base = OTHER; note = 5-[N-(1-naphthylmethyl)carboxamide]-2 prime-deoxyuridine (NapdU)
ccctctgtcaacctccacacaggcctttgatgcaagtatacagtggaaga

SEQ ID NO: 14 - DNA, 50 bp, synthetic construct
  misc_feature 1..50: note = Description of Artificial Sequence: Synthetic oligonucleotide
  source 1..50: mol_type = other DNA
  modified_base 8: mod_base = OTHER; note = 5-[N-(1-naphthylmethyl)carboxamide]-2 prime-deoxyuridine (NapdU)
  modified_base 10: mod_base = OTHER; note = 5-[N-(1-naphthylmethyl)carboxamide]-2 prime-deoxyuridine (NapdU)
  modified_base 16: mod_base = OTHER; note = 5-[N-(1-naphthylmethyl)carboxamide]-2 prime-deoxyuridine (NapdU)
  modified_base 23..24: mod_base = OTHER; note = 5-[N-(1-naphthylmethyl)carboxamide]-2 prime-deoxyuridine (NapdU)
  modified_base 29: mod_base = OTHER; note = 5-[N-(1-naphthylmethyl)carboxamide]-2 prime-deoxyuridine (NapdU)
  modified_base 31: mod_base = OTHER; note = 5-[N-(1-naphthylmethyl)carboxamide]-2 prime-deoxyuridine (NapdU)
  modified_base 37: mod_base = OTHER; note = 5-[N-(1-naphthylmethyl)carboxamide]-2 prime-deoxyuridine (NapdU)
  modified_base 44: mod_base = OTHER; note = 5-[N-(1-naphthylmethyl)carboxamide]-2 prime-deoxyuridine (NapdU)
ccctccctatcgcaatgacacattggggtctcgaggtgcgcgatcgaaga

SEQ ID NO: 15 - protein, 9 aa, Homo sapiens
EHSISRSLL

SEQ ID NO: 16 - protein, 9 aa, synthetic construct
  REGION 1..9: note = Description of Artificial Sequence: Synthetic peptide
YGGWGGWGP

SEQ ID NO: 17 - protein, 4 aa, Homo sapiens
PFPP

SEQ ID NO: 18 - protein, 4 aa, synthetic construct
  REGION 1..4: note = Description of Artificial Sequence: Synthetic peptide
AFAA

SEQ ID NO: 19 - protein, 4 aa, synthetic construct
  REGION 1..4: note = Description of Artificial Sequence: Synthetic peptide
WFRW

SEQ ID NO: 20 - protein, 20 aa, Homo sapiens
SLHGSEADTLRKVLVEVLAD

SEQ ID NO: 21 - protein, 12 aa, Homo sapiens
VEVLADPLDHRD

SEQ ID NO: 22 - protein, 20 aa, Homo sapiens
PLDHRDAGDLWFPGESESFE

SEQ ID NO: 23 - protein, 12 aa, Homo sapiens
ESESFEDAHVEH

SEQ ID NO: 24 - protein, 20 aa, Homo sapiens
DAHVEHSISRSLLEGEIPFP

SEQ ID NO: 25 - protein, 6 aa, Homo sapiens
DAHVEH

SEQ ID NO: 26 - protein, 14 aa, Homo sapiens
SISRSLLEGEIPFP

SEQ ID NO: 27 - protein, 12 aa, Homo sapiens
SRSLLEGEIPFP

SEQ ID NO: 28 - protein, 6 aa, synthetic construct
  REGION 1..6: note = Description of Artificial Sequence: Synthetic 6xHis tag
HHHHHH

(19) United States
(12) Patent Application Publication
(10) Pub. No.: US 2024/0166742 A1
(43) Pub. Date: May 23, 2024

(54) MOLECULES THAT BIND TO AND STABILIZE TRIGGERING RECEPTOR EXPRESSED ON MYELOID CELLS 2 (TREM2)

(71) Applicant: Novartis AG, Basel (CH)

(72) Inventors: Karl Welzenbach, Huningue (FR); Dominik Feuerbach, Müllheim (DE)

(21) Appl. No.: 18/363,237

(22) Filed: Aug. 1, 2023

Related U.S. Application Data
(63) Continuation of application No. 16/446,066, filed on Jun. 19, 2019, now abandoned.
Application No. 16/446,066 is a continuation of application No. 15/239,331, filed on Aug. 17, 2016, now abandoned.
(60) Provisional application No. 62/310,227, filed on Mar. 18, 2016.
(60) Provisional application No. 62/209,403, filed on Aug. 25, 2015.

(51) Int. Cl. C07K 16/28; A61K 39/395; C07K 16/46; C12N 15/115; G01N 33/566

(52) U.S. Cl. CPC C07K 16/2803; A61K 39/3955; C07K 16/468; C12N 15/115; G01N 33/566; C07K 2317/31; C07K 2317/52; C07K 2317/732; C07K 2317/734; C12N 2310/16; G01N 2333/705

(57) ABSTRACT

Provided herein are molecules that bind to and stabilize triggering receptor expressed on myeloid cells 2 (TREM2) and methods of using these molecules.

### PRIORITY CLAIM

This application is a continuation of Ser. No. 16/446,066, filed Jun. 19, 2019, which is a continuation of Ser. No. 15/239,331, filed Aug. 17, 2016, now abandoned, which claims priority to and the benefit under 35 USC § 119(e) to U.S. Provisional Patent Application No. 62/209,403, filed on Aug. 25, 2015; and U.S. Provisional Patent Application No. 62/310,227, filed on Mar. 18, 2016, all of which are hereby incorporated by reference in their entirety.

### SEQUENCE LISTING

The instant application contains a Sequence Listing which has been submitted electronically in XML format and is hereby incorporated by reference in its entirety. Said XML copy, created on Dec. 20, 2023, is named N2067-720621_SL.xml and is 44,212 bytes in size.

### TECHNICAL FIELD

The present invention provides molecules that bind to and stabilize triggering receptor expressed on myeloid cells 2 (TREM2) and methods of using these molecules.

### BACKGROUND

Triggering receptors expressed on myeloid cells or “TREMs” are a group of transmembrane glycoproteins that are expressed on different types of myeloid cells, such as mast cells, monocytes, macrophages, dendritic cells, and neutrophils. TREMs have an immunoglobulin (Ig)-type fold in their extracellular domain and thus belong to the immunoglobulin superfamily (IgSF). TREM receptors contain a short intracellular domain, but lack docking motifs for signaling mediators and require adapter proteins, such as DAP12 (DNAX-activating protein of 12 kDa) for cell activation. Two members of TREMs have been reported: TREM1 and TREM2, both of which play an important role in immune and inflammatory responses.

TREM2 is expressed on macrophages, dendritic cells, osteoclasts, microglia, lung epithelial cells and hepatocarcinoma cells, but absent from myeloid cells in the blood. TREM2 physically associates with DAP12, which acts as a signaling adaptor protein for TREM2 and a number of other cell surface receptors. The cytoplasmic domain of DAP12 contains an immunoreceptor tyrosine activation motif (ITAM) (Wunderlich, J. Biol. Chem. 288, 33027-33036, 2013). After activation of the interacting receptor, DAP12 undergoes phosphorylation at the two conserved ITAM tyrosine residues by Src kinases. Subsequent recruitment and activation of the Syk protein kinase trigger downstream signaling pathways, including the activation of mitogen-activated protein kinase (MAPK) and phospholipase Cγ (PLCγ).

TREM2 can be activated by lipopolysaccharides (LPS), heat shock protein 60, neuritic debris, bacteria, and a broad array of anionic and zwitterionic lipids, e.g. phosphatidic acid (PA), phosphatidylglycerol (PG), phosphatidylserine (PS), phosphatidylinositol (PI), phosphatidylcholine (PC) and sphingomyelin. TREM2 activation increases phagocytic capacity of microglia and macrophages, reduces the release of pro-inflammatory cytokines and limits TLR signaling. TREM2 sustains microglial survival by synergizing with CSF-1 receptor signaling. Further, TREM2 interacts with Plexin-A1 regulating cellular adhesion and motility. TREM2 signaling facilitates degradation of ingested prey and is crucial for lipid metabolism, myelin uptake and intracellular breakdown.

### SUMMARY OF THE INVENTION

Provided herein are molecules, e.g., polypeptides, antibodies, antibody fragments, fusobodies, small molecule weight compounds, modified oligonucelotides, macrocyclic molecules, or aptamers, that specifically bind to the extracellular domain of TREM2 protein (e.g., human TREM2 protein) and stabilize the TREM2 protein (e.g., human TREM2 protein). Those TREM2-binding molecules (e.g., human TREM2-binding molecules) can (i) reduce or inhibit the shedding of the TREM2 ectodomain; (ii) stabilize the TREM2 protein on the cell surface; and/or (iii) maintain or increase TREM2 functions, such as binding to its cognate ligands, intracellular signaling, increasing phagocytosis, and facilitating degradation of phagocytic material and promote TREM2-dependent downstream regulatory functions. Since dysfunctional human TREM2 or absent surface human TREM2 was associated with human neuroinflammatory and neurodegenerative pathologies, the TREM2-stabilizing molecules (e.g., human TREM2-binding molecules) described herein can be used to treat, prevent, or diagnose a neuroinflammatory or neurodegenerative disease such as Alzheimer's disease, frontotemporal dementia, Parkinson's disease, amyotrophic lateral sclerosis, Nasu-Hakola disease, multiple sclerosis, amyotrophic lateral sclerosis (ALS), anti-NMDA receptor encephalitis, autism, brain lupus (NP-SLE), chemo-induced peripheral neuropathy (CIPN), postherapeutic neuralgia, chronic inflammatory demyelinating polyneuropathy (CIDP), epilepsy, Guillain-Barré Syndrom (GBS), inclusion body myositis, lysosomal storage diseases, e.g., sphingomyelinlipidose (Niemann-Pick C) and mucopolysaccharidose II/IIIB, metachromatic leukodystrophy, multifocal motor neuropathy, Myasthenia Gravis, Neuro-Behcet's Disease, neuromyelitis optica (NMO), optic neuritis, polymyositis, dermatomyositis, Rasmussen's encephalitis, Rett's Syndrome, stroke, transverse myelitis, traumatic brain injury, spinal cord injury, viral encephalitis, or bacterial meningitis. The TREM2-binding molecules (e.g., human TREM2-binding molecules) described herein are also suitable for treating, preventing or diagnosing autoimmune, inflammatory, or malignant disorders mediated by or associated with extensive proteolytic cleavage of TREM2 or cells expressing aberrant or mutated variants of the TREM2 receptor. Also provided herein are methods of diagnosing and/or treating TREM2-associated diseases (e.g., human TREM2-associated diseases) using the TREM2-binding molecules (e.g., human TREM2-binding molecules) disclosed herein.

In one aspect, provided herein are methods of treating a disease associated with TREM2 loss of function (e.g., human TREM2 loss of function) in a subject in need thereof by administering to the subject a therapeutically effective amount of a molecule that specifically bind to the extracellular domain of TREM2 protein (e.g., human TREM2 protein) and stabilize the TREM2 protein (e.g., human TREM2 protein). Such methods can include one or more of the following steps: (1) assaying the cell surface TREM2 level (e.g., human TREM2 level) in a sample obtained from a subject; (2) selecting a subject whose cell surface TREM2 level (e.g., human TREM2 level) is lower than a reference level, wherein the reference level is the cell surface TREM2 level (e.g., human TREM2 level) in a sample obtained from a healthy subject; and (3) administering to the selected subject a therapeutically effective amount of a molecule that specifically binds to the extracellular domain of TREM2 protein (e.g., human TREM2 protein) and stabilizes the TREM2 protein (e.g., human TREM2 protein). In some embodiments, such methods further include administering a second agent to the subject. The cell surface TREM2 level (e.g., human TREM2 level) in a sample can be determined by an assay selected from flow cytometry, immunohistochemistry, Western blotting, immunofluorescent assay, radioimmunoassay (RIA), enzyme-linked immunosorbent assay (ELISA), homogeneous time resolved fluorescence (HTRF), or positron emission tomography (PET). In some embodiments, the sample comprises cerebrospinal fluid and its cellular components. In some embodiments, the disease associated with TREM2 loss of function (e.g., human TREM2 loss of function) is a neuroinflammatory or neurodegenerative disease such as Alzheimer's disease, frontotemporal dementia, Parkinson's disease, amyotrophic lateral sclerosis, Nasu-Hakola disease, multiple sclerosis, amyotrophic lateral sclerosis (ALS), anti-NMDA receptor encephalitis, autism, brain lupus (NP-SLE), chemo-induced peripheral neuropathy (CIPN), postherapeutic neuralgia, chronic inflammatory demyelinating polyneuropathy (CIDP), epilepsy, Guillain-Barre Syndrom (GBS), inclusion body myositis, lysosomal storage diseases, e.g., sphingomyelinlipidose (Niemann-Pick C) and mucopolysaccharidose II/IIIB, metachromatic leukodystrophy, multifocal motor neuropathy, Myasthenia Gravis, Neuro-Behcet's Disease, neuromyelitis optica (NMO), optic neuritis, polymyositis, dermatomyositis, Rasmussen's encephalitis, Rett's Syndrome, stroke, transverse myelitis, traumatic brain injury, spinal cord injury, viral encephalitis, or bacterial meningitis. In some embodiments, the disease associated with TREM2 loss of function (e.g., human TREM2 loss of function) is a neurodegenerative disease selected from Alzheimer's disease, frontotemporal dementia, Parkinson's disease, amyotrophic lateral sclerosis, or Nasu-Hakola disease. In some embodiments, the TREM2-binding molecules (e.g., human TREM2-binding molecules) stabilize the TREM2 protein (e.g., human TREM2 protein) on the cell surface of a TREM2 expressing cell selected from a macrophage, dendritic cell, osteoclast, microglia, lung epithelial cell, or hepatocarcinoma cell. In some embodiments, the TREM2-binding molecule (e.g., human TREM2-binding molecule) is administered to the subject through an oral, intravenous, intracranial, intrathecal, subcutaneous, or intranasal route. In some embodiments, the TREM2-binding molecule (e.g., human TREM2-binding molecule) is an antibody, e.g., a monoclonal antibody.

In another aspect, provided herein are molecules that specifically bind to the extracellular domain of TREM2 protein (e.g., human TREM2 protein) and stabilize the TREM2 protein (e.g., human TREM2 protein). In some embodiments, these molecules stabilize the TREM2 protein (e.g., human TREM2 protein) on the cell surface of a TREM2 expressing cell such as a macrophage, dendritic cell, osteoclast, microglia, lung epithelial cell, or hepatocarcinoma cell. In some embodiments, these molecules reduce proteolytic shedding of the ectodomain of the TREM2 protein (e.g., human TREM2 protein). In some embodiments, those TREM2-binding molecules (e.g., human TREM2-binding molecules) are polypeptides, e.g., antibodies or antigen-binding fragments thereof, e.g., monoclonal antibodies. In some embodiments, those TREM2-binding molecules (e.g., human TREM2-binding molecules) are aptamers, e.g., slow off-rate modified aptamers. In some embodiments, those TREM2-binding molecules (e.g., human TREM2-binding molecules) are low molecular weight compounds.

In some embodiments, provided herein are molecules that specifically bind to the extracellular domain of human TREM2. For example, such molecules bind to the extracellular domain of human TREM2 that comprises the amino acid residues 14 to 174 of SEQ ID NO: 1, the amino acid residues 14 to 168 of SEQ ID NO: 3, or the amino acid residues 14 to 171 of SEQ ID NO: 4.

In some embodiments, provided herein are molecules that specifically bind to a stalk region of TREM2 (e.g., human TREM2 protein). For example, such molecules bind to a stalk region of human TREM2 that comprises an amino acid sequence of any of SEQ ID NO: 7, 8, or 9.

In some embodiments, provided herein are monoclonal antibodies or antigen binding fragments thereof that specifically bind to the extracellular domain of TREM2 protein (e.g., human TREM2 protein) and stabilize the TREM2 protein (e.g., human TREM2 protein). For example, the monoclonal antibodies or antigen binding fragments thereof bind to the extracellular domain of human TREM2 that comprises the amino acid residues 14 to 174 of SEQ ID NO: 1, the amino acid residues 14 to 168 of SEQ ID NO: 3, or the amino acid residues 14 to 171 of SEQ ID NO: 4. In some embodiments, the monoclonal antibodies or antigen binding fragments thereof specifically bind to a stalk region of TREM2 protein (e.g., human TREM2 protein). For example, the monoclonal antibodies or antigen binding fragments thereof bind to a stalk region of human TREM2 that comprises an amino acid sequence of any of SEQ ID NO: 7, 8, or 9. In some embodiments, the TREM2 monoclonal antibodies are human or humanized antibody. In some embodiments, the antigen binding fragment is a Fab, F(ab′)2, Fv fragments, scFv, minibody, or diabody.

In some embodiments, the TREM2-binding molecule (e.g., human TREM2-binding molecule) is a bispecific antibody. In some embodiments, the bispecific antibody specifically binds to human TREM2 and human DAP12.

In some embodiments, the TREM2-binding molecule (e.g., human TREM2-binding molecule) comprises an Fc region. In some embodiments, the Fc region has reduced antibody-dependent cellular cytotoxicity (ADCC) or complement-dependent cytotoxicity (CDC) activity when compared to the parental antibody. In some embodiments, the Fc region is selected from a modified IgG1 Fc region, an IgG2 Fc region, an IgG4 Fc region, or an IgG2/IgG4 hybrid Fc region.

Provided herein are also nucleic acid encoding such monoclonal antibodies or antigen binding fragments thereof, and vectors and host cells comprising nucleic acid encoding such monoclonal antibodies or antigen binding fragments thereof.

In another aspect, provided herein are pharmaceutical compositions comprising one or more of the TREM2-binding molecules (e.g., human TREM2-binding molecules) described herein and a pharmaceutically acceptable carrier.

In another aspect, provided herein are molecules for use in the treatment of a disease associated with TREM2 loss of function (e.g., human TREM2 loss of function). In some embodiments, these molecules specifically bind to the extracellular domain of TREM2 protein (e.g., human TREM2 protein) and stabilize the TREM2 protein (e.g., human TREM2 protein). In some embodiments, the molecules stabilize the TREM2 protein (e.g., human TREM2 protein) on the cell surface of a TREM2-expressing cell selected from a macrophage, dendritic cell, osteoclast, microglia, lung epithelial cell, or hepatocarcinoma cell. In some embodiments, the molecules reduce shedding of the ectodomain of the TREM2 protein (e.g., human TREM2 protein). In some embodiments, the disease associated with TREM2 loss of function (e.g., human TREM2 loss of function) is a neuroinflammatory or neurodegenerative disease such as Alzheimer's disease, frontotemporal dementia, Parkinson's disease, amyotrophic lateral sclerosis, Nasu-Hakola disease, multiple sclerosis, amyotrophic lateral sclerosis (ALS), anti-NMDA receptor encephalitis, autism, brain lupus (NP-SLE), chemo-induced peripheral neuropathy (CIPN), postherapeutic neuralgia, chronic inflammatory demyelinating polyneuropathy (CIDP), epilepsy, Guillain-Barré Syndrom (GBS), inclusion body myositis, lysosomal storage diseases, e.g., sphingomyelinlipidose (Niemann-Pick C) and mucopolysaccharidose II/IIIB, metachromatic leukodystrophy, multifocal motor neuropathy, Myasthenia Gravis, Neuro-Behcet's Disease, neuromyelitis optica (NMO), optic neuritis, polymyositis, dermatomyositis, Rasmussen's encephalitis, Rett's Syndrome, stroke, transverse myelitis, traumatic brain injury, spinal cord injury, viral encephalitis, or bacterial meningitis. In some embodiments, the disease associated with TREM2 loss of function (e.g., human TREM2 loss of function) is a neurodegenerative disease selected from Alzheimer's disease, frontotemporal dementia, Parkinson's disease, amyotrophic lateral sclerosis, or Nasu-Hakola disease. In some embodiments, the molecules bind to the extracellular domain of human TREM2, for example, the extracellular domain of human TREM2 comprising the amino acid residues 14 to 174 of SEQ ID NO: 1, the amino acid residues 14 to 168 of SEQ ID NO: 3, or the amino acid residues 14 to 171 of SEQ ID NO: 4. In some embodiments, the molecules bind to a stalk region of human TREM2, for example, a stalk region of human TREM2 comprising an amino acid sequence of any of SEQ ID NO: 7, 8, or 9.

### Definitions

As used in the specification and claims, the singular form “a”, “an” and “the” include plural references unless the context clearly dictates otherwise. For example, the term “a cell” includes a plurality of cells, including mixtures thereof.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about.” It also is to be understood, although not always explicitly stated, that the reagents described herein are merely examples and that equivalents of such are known in the art.

As used herein, “TREM2” (also known as “triggering receptor expressed on myeloid cells 2”, TREM-2, TREM2a, TREM2b, or TREM2c) refers to a glycoprotein encoded by the TREM2 gene. Human TREM2 belongs to the immunoglobulin superfamily (IgSF), and includes a signal peptide, a single V-type immunoglobulin domain (IgV), a stalk region, a transmembrane domain, and a cytoplasmic tail. The human TREM2 gene is mapped to chromosomal location 6p21.1, and the genomic sequence of human TREM2 gene can be found in GenBank at NC_000006.12. Due to alternative splicing, there are at least three human TREM2 isoforms. The term “human TREM2” is used to refer to any isoform of human TREM2. The protein and mRNA sequences for the longest human TREM2 isoform (isoform 1) are:

Triggering receptor expressed on myeloid cells 2 precursor isoform 1 precursor
[Homo sapiens] (NP_061838.1)
(SEQ ID NO: 1)
MEPLRLLILLFVTELSGAHNTTVFQGVAGQSLQVSCPYDSMKHWGRRKAWC
RQLGEKGPCQRVVSTHNLWLLSFLRRWNGSTAITDDTLGGTLTITLRNLQPHDAGLY
QCQSLHGSEADTLRKVLVEVLADPLDHRDAGDLWFPGESESFEDAHVEHSISRSLLE
GEIPFPPTSILLLLACIFLIKILAASALWAAAWHGQKPGTHPPSELDCGHDPGYQLQTL
PGLRDT
Homo sapiens triggering receptor expressed on myeloid cells 2 (TREM2), transcript
variant 1, mRNA (NM_018965.3)
(SEQ ID NO: 2)
gggcagcgcc tgacatgcct gatcctctct tttctgcagt tcaagggaaa gacgagatct tgcacaaggc
actctgcttc tgcccttggc tggggaaggg tggcatggag cctctccggc tgctcatctt actctttgtc acagagctgt
ccggagccca caacaccaca gtgttccagg gcgtggcggg ccagtccctg caggtgtctt gcccctatga ctccatgaag
cactggggga ggcgcaaggc ctggtgccgc cagctgggag agaagggccc atgccagcgt gtggtcagca cgcacaactt
gtggctgctg tccttcctga ggaggtggaa tgggagcaca gccatcacag acgataccct gggtggcact ctcaccatta
cgctgcggaa tctacaaccc catgatgcgg gtctctacca gtgccagagc ctccatggca gtgaggctga caccctcagg
aaggtcctgg tggaggtgct ggcagacccc ctggatcacc gggatgctgg agatctctgg ttccccgggg agtctgagag
cttcgaggat gcccatgtgg agcacagcat ctccaggagc ctcttggaag gagaaatccc cttcccaccc acttccatcc
ttctcctcct ggcctgcatc tttctcatca agattctagc agccagcgcc ctctgggctg cagcctggca tggacagaag
ccagggacac atccacccag tgaactggac tgtggccatg acccagggta tcagctccaa actctgccag ggctgagaga
cacgtgaagg aagatgatgg gaggaaaagc ccaggagaag tcccaccagg gaccagccca gcctgcatac ttgccacttg
gccaccagga ctccttgttc tgctctggca agagactact ctgcctgaac actgcttctc ctggaccctg gaagcaggga
ctggttgagg gagtggggag gtggtaagaa cacctgacaa cttctgaata ttggacattt taaacactta caaataaatc
caagactgtc atatttagct ggataaaaaa aaaaaaaaaa aaaaaa

The amino acid sequences of human TREM2 isoform 2 (SEQ ID NO: 3) and isoform 3 (SEQ ID NO: 4) are shown in FIG. 1B. In some embodiments, TREM2 protein also encompasses proteins that have over its full length at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with any of SEQ ID No: 1, 3, or 4, wherein such proteins still have the ligand binding, intracellular signaling, facilitating phagocytosis and degradation of phagocytic material, and other regulatory function of TREM2. The sequences of murine, cyno, and other animal TREM2 proteins are known in the art (for example, NP_112544.1 and NP_001259007.1 for murine TREM2 protein).

The term “extracellular domain” refers to the portion of a transmembrane protein that is exposed on the extracellular side of a lipid bilayer of a cell. Methods for determining the ectodomain of a protein are known in the art (Singer (1990); High et al. (1993), and McVector software, Oxford Molecular). For example, the extracellular domain of human TREM2 protein can include the amino acid residues 14 to 174 of SEQ ID NO: 1 (isoform 1), the amino acid residues 14 to 168 of SEQ ID NO: 3 (isoform 2), or the amino acid residues 14 to 171 of SEQ ID NO: 4 (isoform 3).

The term “ectodomain” of TREM2 refers to a portion of the extracellular domain of TREM2 that is released after sheddase cleavage.

The term “stalk region” of TREM2 refers to a portion of the extracellular domain of TREM2 that connects the V-type immunoglobulin (IgV) domain and the transmembrane domain. For example, the stalk region of human TREM2 protein can include an amino acid sequence of any of SEQ ID NO: 7, 8, or 9.

The term “transmembrane domain” refers to the portion of a transmembrane protein that spans the lipid bilayer of a cell. Methods for determining the transmembrane domain of a protein are known in the art (Elofsson et al. (2007) Annu. Rev. Biochem. 76:125-140; Bernsel et al. (2005) Protein Science 14:1723-1728).

The terms “cytoplasmic domain” and “cytoplasmic tail” are used interchangeably and refer to the portion of a transmembrane protein that is on the cytoplasmic side of the lipid bilayer of a cell. Methods for determining the cytoplasmic tail of a protein are known in the art (Elofsson et al. (2007) and Bernsel et al. (2005)).

The term “stabilize” as used herein refers to the maintenance or restoration of TREM2 cell surface level in a TREM2-expressing cell to a normal level, e.g., the TREM2 level in a corresponding TREM2-expressing cell in a healthy subject without inflammatory or neurodegerative disease, or to an increased level, e.g., an increased TREM2 level in a subject after a treatment compared to the TREM2 level in the same subject before the treatment. This may be accomplished by reducing or inhibiting the shedding of TREM2 ectodomain, or by increasing cell surface expression of TREM2. TREM2 cell surface level can be assessed by TREM2 FACS staining or by TREM2 cell surface immunoprecipitation or by the reduction of soluble TREM2 over time. TREM2 cell surface expression can also be detected by, enzyme-linked immunosorbent assay (ELISA), radioimmunoassay (RIA), bioassays (e.g., increase in phagocytosis), Western Blot assay, flow cytometry, immunohistochemistry, immunofluorescent assay, homogeneous time resolved fluorescence (HTRF), or positron emission tomography (PET).

The term “antibody,” as used herein, refers to a protein, or polypeptide sequence derived from an immunoglobulin molecule that specifically binds to an antigen. Antibodies can be polyclonal or monoclonal, multiple or single chain, or intact immunoglobulins, and may be derived from natural sources or from recombinant sources. For example, a naturally occurring IgG antibody can be a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. An antibody can be a monoclonal antibody, human antibody, humanized antibody, camelised antibody, or chimeric antibody. The antibodies can be of any isotype (e.g., IgG, IgE, IgM, IgD, IgA and IgY), class (e.g., IgG1, IgG2, IgG3, IgG4, IgA1 and IgA2) or subclass.

The term “antibody,” as used herein, also includes antibody fragment or antigen-binding fragment. The term “antibody fragment” or “antigen-binding fragment” refers to at least one portion of an antibody, that retains the ability to specifically interact with (e.g., by binding, steric hinderance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide brudge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies). The term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The terms “complementarity determining regions” or “CDRs” as used herein, refer to the amino acid residues of an antibody or antigen-binding fragment that are responsible for antigen binding specificity and/or affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme), or a combination thereof.

The term “epitope” includes any protein determinant capable of specific binding to an immunoglobulin or otherwise interacting with a molecule. Epitopic determinants generally consist of chemically active surface groupings of molecules such as amino acids or carbohydrate or sugar side chains and can have specific three-dimensional structural characteristics, as well as specific charge characteristics. An epitope may be “linear” or “conformational.” Conformational and linear epitopes are distinguished in that the binding to the former but not the latter is lost in the presence of denaturing solvents.

The term “monovalent antibody” as used herein, refers to an antibody that binds to a single epitope on a target molecule.

The term “bivalent antibody” as used herein, refers to an antibody that binds to two epitopes on at least two identical target molecules. The bivalent antibody may also crosslink the target molecules to one another. A “bivalent antibody” also refers to an antibody that binds to two different epitopes on at least two identical target molecules.

The term “multivalent antibody” refers to a single binding molecule with more than one valency, where “valency” is described as the number of antigen-binding moieties present per molecule of an antibody construct. As such, the single binding molecule can bind to more than one binding site on a target molecule. Examples of multivalent antibodies include, but are not limited to bivalent antibodies, trivalent antibodies, tetravalent antibodies, pentavalent antibodies, and the like, as well as bispecific antibodies and biparatopic antibodies. For example, for TREM2, the multivalent antibody (e.g., a TREM2 biparatopic antibody) has a binding moiety for two domains of TREM2, respectively.

The term “multivalent antibody” also refers to a single binding molecule that has more than one antigen-binding moiety for two separate target molecules. For example, an antibody that binds to TREM2 and a second target molecule that is not TREM2. In one embodiment, a multivalent antibody is a tetravalent antibody that has four epitope binding domains. A tetravalent molecule may be bispecific and bivalent for each binding site on that target molecule.

The term “bispecific antibody” as used herein, refers to an antibody that binds to two or more different epitopes. In some embodiments, a bispecific antibody binds to two different targets. In some embodiments, a bispecific antibody binds to two different epitopes on a single target molecule. An antibody that binds to two different epitopes on a single target molecule is also known as a “biparatopic antibody.”

The phrases “monoclonal antibody” or “monoclonal antibody composition” as used herein refers to polypeptides, including antibodies, bispecific antibodies, etc., that have substantially identical amino acid sequence or are derived from the same genetic source. This term also includes preparations of antibody molecules of single molecular composition. A monoclonal antibody composition displays a single binding specificity and affinity for a particular epitope.

The phrase “human antibody,” as used herein, includes antibodies having variable regions in which both the framework and CDR regions are derived from sequences of human origin. Furthermore, if the antibody contains a constant region, the constant region is also derived from such human sequences, e.g., human germline sequences, or mutated versions of human germline sequences or antibody containing consensus framework sequences derived from human framework sequences analysis, for example, as described in Knappik, et al. (2000. J Mol Biol 296, 57-86). The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g., Sequences of Proteins of Immunological Interest, U.S. Department of Health and Human Services (1991), eds. Kabat et al.; Al Lazikani et al., (1997) J. Mol. Bio. 273:927 948); Kabat et al., (1991) Sequences of Proteins of Immunological Interest, 5th edit., NIH Publication no. 91-3242 U.S. Department of Health and Human Services; Chothia et al., (1987) J. Mol. Biol. 196:901-917; Chothia et al., (1989) Nature 342:877-883; and Al-Lazikani et al., (1997) J. Mal. Biol. 273:927-948.

The human antibodies of the invention may include amino acid residues not encoded by human sequences (e.g., mutations introduced by random or site-specific mutagenesis in vitro or by somatic mutation in vivo, or a conservative substitution to promote stability or manufacturing). However, the term “human antibody” as used herein, is not intended to include antibodies in which CDR sequences derived from the germline of another mammalian species, such as a mouse, have been grafted onto human framework sequences.

The phrase “recombinant human antibody” as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “Fc region” as used herein refers to a polypeptide comprising the CH3, CH2 and at least a portion of the hinge region of a constant domain of an antibody. Optionally, an Fc region may include a CH4 domain, present in some antibody classes. An Fc region, may comprise the entire hinge region of a constant domain of an antibody. In one embodiment, the invention comprises an Fc region and a CH1 region of an antibody. In one embodiment, the invention comprises an Fc region CH3 region of an antibody. In another embodiment, the invention comprises an Fc region, a CH1 region and a Ckappa/lambda region from the constant domain of an antibody. In one embodiment, a binding molecule of the invention comprises a constant region, e.g., a heavy chain constant region. In one embodiment, such a constant region is modified compared to a wild-type constant region. That is, the polypeptides of the invention disclosed herein may comprise alterations or modifications to one or more of the three heavy chain constant domains (CH1, CH2 or CH3) and/or to the light chain constant region domain (CL). Example modifications include additions, deletions or substitutions of one or more amino acids in one or more domains. Such changes may be included to optimize effector function, half-life, etc.

As used herein, the term “affinity” refers to the strength of interaction between antibody and antigen at single antigenic sites. Within each antigenic site, the variable region of the antibody “arm” interacts through weak non-covalent forces with the antigen at numerous sites; the more interactions, the stronger the affinity. As used herein, the term “high affinity” for an IgG antibody or fragment thereof (e.g., a Fab fragment) refers to an antibody having a knock down of 10⁻⁸ M or less, 10⁻⁹ M or less, or 10⁻¹⁰ M, or 10⁻¹¹ M or less, or 10⁻¹² M or less, or 10⁻¹³ M or less for a target antigen. However, high affinity binding can vary for other antibody isotypes. For example, high affinity binding for an IgM isotype refers to an antibody having a knock down of 10⁻⁷ M or less, or 10⁻⁸ M or less.

As used herein, the term “avidity” refers to an informative measure of the overall stability or strength of the antibody-antigen complex. It is controlled by three major factors: antibody epitope affinity; the valency of both the antigen and antibody; and the structural arrangement of the interacting parts. Ultimately these factors define the specificity of the antibody, that is, the likelihood that the particular antibody is binding to a precise antigen epitope.

The term “binding specificity” as used herein refers to the ability of an individual antibody combining site to react with one antigenic determinant and not with a different antigenic determinant. The combining site of the antibody is located in the Fab portion of the molecule and is constructed from the hypervariable regions of the heavy and light chains. Binding affinity of an antibody is the strength of the reaction between a single antigenic determinant and a single combining site on the antibody. It is the sum of the attractive and repulsive forces operating between the antigenic determinant and the combining site of the antibody.

The term “aptamer,” as used herein, refers to a polynucleotide or polypeptide molecule that, through its ability to adopt a specific three dimensional conformation, binds to a protein target and modifies the protein target or the functional activity or stability of the protein target.

As used herein, the terms “slow off-rate,” or “slow rate of dissociation,” or “slow dissociation rate,” refers to the time it takes for an aptamer to begin to dissociate from a target molecule. This can be expressed as a half-life, t_{1/2}, or the point at which 50% of the aptamer/target complex has dissociated. The off-rate or dissociation rate of a slow off-rate aptamer, expressed as t_{1/2} values, can be ≥about 15 minutes, ≥about 30 minutes, ≥about 60 minutes, ≥about 90 minutes, ≥about 120 minutes, ≥about 150 minutes, ≥about 180 minutes, ≥about 210 minutes, and ≥about 240 minutes.

The term “treat” and “treatment” refer to both therapeutic treatment and prophylactic or preventive measures, wherein the object is to prevent or slow down an undesired physiological change or disorder. For purpose of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment.

The term “subject” refers to an animal, human or non-human, to whom treatment according to the methods of the present invention is provided. Veterinary and non-veterinary applications are contemplated. The term includes, but is not limited to, mammals, e.g., humans, other primates, pigs, rodents such as mice and rats, rabbits, guinea pigs, hamsters, cows, horses, cats, dogs, sheep and goats. Typical subjects include humans, farm animals, and domestic pets such as cats and dogs.

An “effective amount” refers to an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A “therapeutically effective amount” of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. Percentage of “sequence identity” can be determined by comparing two optimally aligned sequences over a comparison window, where the fragment of the amino acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical amino acid residue occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. An isolated antibody is substantially free of other antibodies having different antigenic specificities (e.g., an isolated antibody that specifically binds TREM2 is substantially free of antibodies that specifically bind antigens other than TREM2). An isolated antibody that specifically binds a target molecule may, however, have cross-reactivity to the same antigens from other species, e.g., an isolated antibody that specifically binds TREM2 may bind TREM2 molecules from other species. Moreover, an isolated antibody may be substantially free of other cellular material and/or chemicals.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1A shows exemplary alignment of the amino acid sequences of human TREM2 isoform 1 (SEQ ID NO: 1), Cyno TREM2 isoform 1 (SEQ ID NO: 5), and mouse TREM2 isoform 1 (SEQ ID NO: 6). The stalk region of TREM2 includes the light grey shaded residues. The transmembrane domain of TREM2 includes the yellow shaded residues.

FIG. 1B shows exemplary alignment of the amino acid sequences of human TREM2 isoform 1 (SEQ ID NO: 1), isoform 2 (SEQ ID NO: 3), and isoform 3 (SEQ ID NO: 4).

FIG. 1C shows exemplary alignment of the amino acid sequences of the stalk regions of human TREM2 isoform 1 (SEQ ID NO: 7), isoform 2 (SEQ ID NO: 8), and isoform 3 (SEQ ID NO: 9).

FIG. 1D shows exemplary alignment of the amino acid sequences of the stalk regions of human TREM2 isoform 1 (SEQ ID NO: 7), Cyno TREM2 isoform 1 (SEQ ID NO: 10), and mouse TREM2 isoform 1 (SEQ ID NO: 11).

FIG. 1E illustrates the structure of TREM2 and its interaction with the signaling adaptor protein DAP12. Mature TREM2 includes a single immunoglobulin (IgSF) domain, a stalk region, a transmembrane (TM) domain, and a cytoplasmic domain.

FIG. 2 is a bar graph showing the specific binding of TREM2 SOMAmers to cellular human TREM2.

FIG. 3 is a line graph showing concentration-dependent binding of different TREM2 SOMAmers to CHO-hTREM2 cells.

FIG. 4 is a line graph showing that TREM2 SOMAmers stabilized cell surface human TREM-2.

FIG. 5A is a bar graph showing that the SOMAmer KX86-94WC stabilized cell surface human TREM2 on CHO-hTREM2 cells.

FIG. 5B is a bar graph showing that the SOMAmer KX86-94WC stabilized cell surface human TREM2 on primary human M2a macrophages.

FIG. 6A is a line graph showing TACE and ADAM10 inhibitors increased surface human TREM2 level in THP1 cells.

FIG. 6B is a bar graph showing TACE and ADAM10 inhibitors reduced formation of soluble fragments of human TREM2 in THP1 cells.

FIG. 6C is a bar graph showing anti-TREM2 antibody stabilized cell surface human TREM2 level in in THP1 cells during PMA activation.

FIG. 7 shows identification of sheddase cleavage site in the stalk region of human TREM2 by serial truncations.

FIG. 8 shows representative high performance liquid chromatography-high resolution mass spectrometry (HPLC-HRMS) data of TACE cleavage of human TREM2 stalk region peptide 78: DAHVEHSISRSLLEGEIPFP (SEQ ID NO: 24). Two main cleavage products of peptide 78 by human TACE were observed: DAHVEH (termed X01, SEQ ID NO: 25) and SISRSLLEGEIPFP (termed X02, SEQ ID NO: 26), suggest that human TACE cleaves the stalk region of human TREM2 isoform 1 between Histidine 157 and Serine 158 of SEQ ID NO: 1.

FIG. 9 is a bar graph showing pre-treatment with an anti-human TREM2 antibody BAF1828, but not with the isotype control BAF108, stabilized human TREM2 cell surface expression in the presence of PMA.

FIG. 10 is a bar graph showing stabilization of human TREM2 at the cell surface after treatment with anti-human TREM2 antibody AF1828 or an inhibitor of TACE and ADAM10 (PI1).

FIG. 11 is a bar graph showing concentration-dependent stabilization of cell surface human TREM2 by anti-human TREM2 antibody AF1828.

FIG. 12 is a line graph showing the time course of cell surface human TREM2 stabilization by anti-human TREM2 antibody AF1828.

FIGS. 13A-13C are bar graphs showing concentration-dependent stabilization of the chimeric human TREM2stalk-TREM1-IGSF switch construct by anti-human TREM2 antibody AF1828 (FIGS. 13A and 13B), but not by the isotype control antibody (FIG. 13C).

### DETAILED DESCRIPTION

Provided herein are molecules, e.g., polypeptides, antibodies, antibody fragments, fusobodies, small molecule weight compounds, modified oligonucelotides, macrocyclic molecules, or aptamers, that specifically bind to the extracellular domain of TREM2 protein (e.g., human TREM2 protein) and stabilize the TREM2 protein (e.g., human TREM2 protein). Those TREM2-binding molecules (e.g., human TREM2-binding molecules) can (i) reduce or inhibit the shedding of the TREM2 ectodomain; (ii) stabilize the TREM2 protein on the cell surface; and/or (iii) maintain or increase TREM2 functions, such as binding to its cognate ligands, intracellular signaling, increasing phagocytosis, and facilitating degradation of phagocytic material and promote TREM2-dependent downstream regulatory functions. Since dysfunctional human TREM2 or absent surface human TREM2 was associated with human neuroinflammatory and neurodegenerative pathologies, the TREM2-stabilizing molecules (e.g., human TREM2-binding molecules) described herein can be used to treat, prevent, or diagnose a neuroinflammatory or neurodegenerative disease such as Alzheimer's disease, frontotemporal dementia, Parkinson's disease, amyotrophic lateral sclerosis, Nasu-Hakola disease, multiple sclerosis, amyotrophic lateral sclerosis (ALS), anti-NMDA receptor encephalitis, autism, brain lupus (NP-SLE), chemo-induced peripheral neuropathy (CIPN), postherapeutic neuralgia, chronic inflammatory demyelinating polyneuropathy (CIDP), epilepsy, Guillain-Barré Syndrom (GBS), inclusion body myositis, lysosomal storage diseases, e.g., sphingomyelinlipidose (Niemann-Pick C) and mucopolysaccharidose II/IIIB, metachromatic leukodystrophy, multifocal motor neuropathy, Myasthenia Gravis, Neuro-Behcet's Disease, neuromyelitis optica (NMO), optic neuritis, polymyositis, dermatomyositis, Rasmussen's encephalitis, Rett's Syndrome, stroke, transverse myelitis, traumatic brain injury, spinal cord injury, viral encephalitis, or bacterial meningitis. The TREM2-binding molecules (e.g., human TREM2-binding molecules) described herein are also suitable for treating, preventing or diagnosing autoimmune, inflammatory, or malignant disorders mediated by or associated with extensive proteolytic cleavage of TREM2 or cells expressing aberrant or mutated variants of the TREM2 receptor. Also provided herein are methods of diagnosing and/or treating TREM2-associated diseases (e.g., human TREM2-associated diseases) using the TREM2-binding molecules (e.g., human TREM2-binding molecules) disclosed herein.

TREM-2 mediates non-phlogistic phagocytosis of bacteria and dying cells and dampens inflammatory responses. Homozygous loss of function of human TREM-2 causes Nasu-Hakola disease (polycystic lipomembranous osteodysplasia with sclerosing leukoencephalopathy, “PLOSL”), or fronto-temporal dementia (FTD)-like syndrome, diseases characterized by bone cysts, neuroinflammation, progressive neurodegeneration and presenile dementia. A heterozygous loss of function mutation R47H of TREM-2 is also an important risk factor for late-onset Alzheimer's disease (AD), with an effect size that is similar to that of the apolipoprotein E ε4 allele. TREM-2 is expressed in the microglia found in the white matter, hippocampus and neocortex, which is partly consistent with the pathological features reported in AD brains, supporting the possible involvement of TREM-2 in AD pathogenesis. Genetic screenings have now also identified heterozygous missense mutations in TREM2 as risk factors for Parkinson's disease (PD), amyotrophic lateral sclerosis (ALS), and fronto-temporal dementia (FTD), in addition to AD (Kleinberger, Sci Transl Med. 2014 Jul. 2; 6(243):243ra86). Thus, functional TREM-2 is required to protect against ageing-related neuroinflammatory and neurodegenerative diseases that cause severe cognitive impairment and dementia.

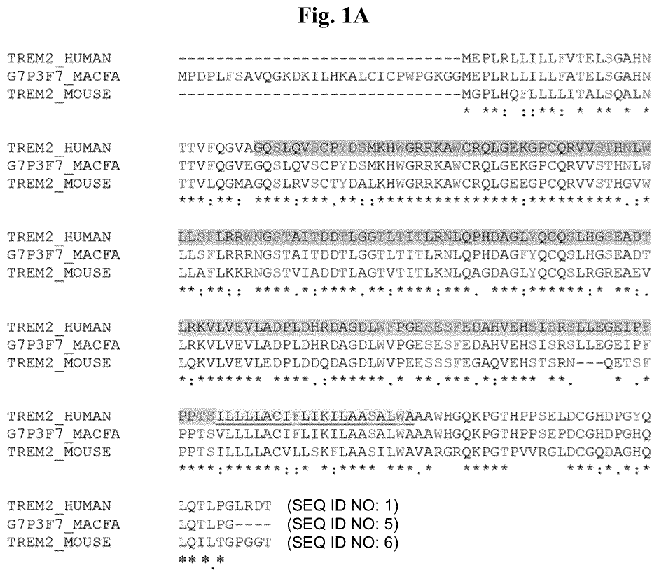

Due to alternative splicing, there are three human TREM2 isoforms, with the isoform 1 being the longest isoform. The amino acid sequences of human TREM2 isoform 1 (SEQ ID NO: 1), human TREM2 isoform 2 (SEQ ID NO: 3), and human TREM2 isoform 3 (SEQ ID NO: 4) were aligned in FIG. 1B. Alignment of the amino acid sequences of the stalk regions of human TREM2 isoform 1 (SEQ ID NO: 7), isoform 2 (SEQ ID NO: 8), and isoform 3 (SEQ ID NO: 9) revealed that the stalk region of human TREM2 isoform 1 shares about 79% sequence identity to the stalk region of human TREM2 isoforms 2 or 3 (FIG. 1C).

The amino acid sequences of human TREM2 isoform 1 (SEQ ID NO: 1), Cyno TREM2 isoform 1 (SEQ ID NO: 5), and mouse TREM2 isoform 1 (SEQ ID NO: 6) were aligned in FIG. 1A. Alignment of the amino acid sequences of the stalk regions of human TREM2 isoform 1 (SEQ ID NO: 7), Cyno TREM2 isoform 1 (SEQ ID NO: 10), and mouse TREM2 isoform 1 (SEQ ID NO: 11) revealed that human TREM2 isoform 1 stalk region shares 98% sequence identity to Cyno TREM2 isoform 1 stalk region, and 69% sequence identity to mouse TREM2 isoform 1 stalk region (FIG. 1D). FIG. 1E illustrates the structure of TREM2 and its interaction with the signaling adaptor protein DAP12.

Identification of Sheddase Cleavage Sites in TREM2

TREM2 undergoes sequential proteolytic processing by ectodomain shedding and intramembrane proteolysis (Wunderlich, J. Biol. Chem. 288, 33027-33036, 2013). During ectodomain shedding, the ectodomain of TREM2 is released by proteases such as members of the ADAM (a disintegrin and metalloproteinase domain containing protein) or BACE (b-site APP cleaving enzyme) family (Kleinberger, Sci Transl Med. 2014 Jul. 2; 6(243):243ra86). After removal of the ectodomain, the remaining membrane-retained fragment is further processed by γ-secretase mediated intramembranous proteolysis. Soluble fragments of TREM2 (sTREM2) produced by ectodomain shedding have been observed in supernatants of dendritic cell cultures as well as in plasma and CSF samples from patients with noninflammatory neurological diseases and multiple sclerosis (Kleinberger, Sci Transl Med. 2014 Jul. 2; 6(243):243ra86).

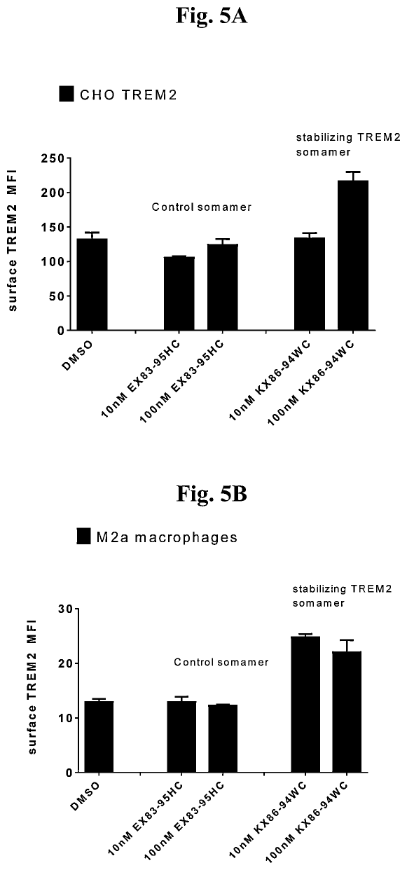

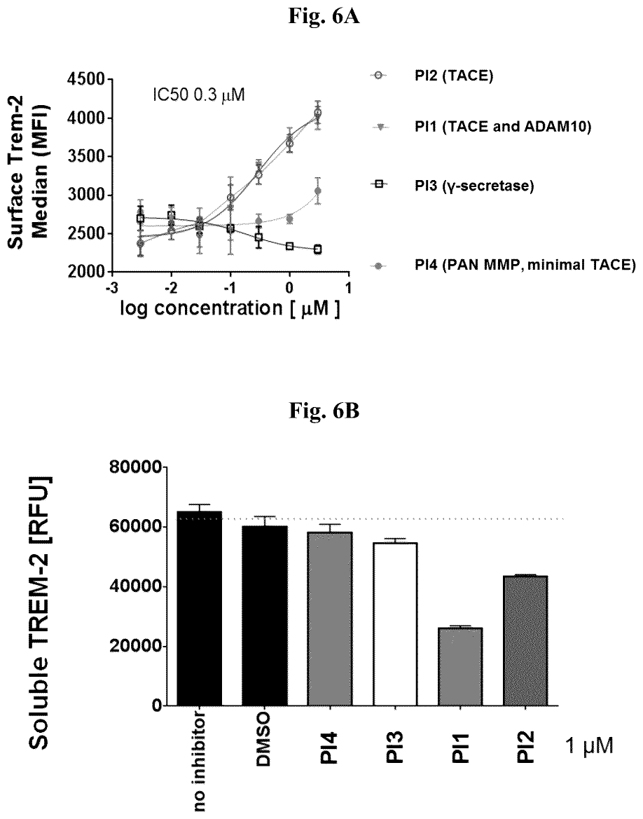

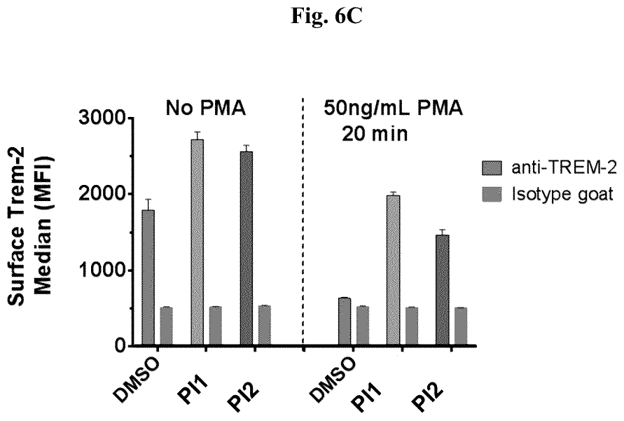

The present disclosure showed that TREM2 is constitutively shed in THP-1 cells, and such shedding was inhibited by both a TACE (ADAM17)-specific inhibitor and a ADAM10/17 inhibitor, indicating that TACE and ADAM10 contribute to TREM2 shedding (FIGS. 6A-C). By generating a series of deletion and substitution TREM2 mutants, two sites within the TREM2 stalk region proximal to the plasma membrane were revealed as possible cleavage or recognition sites for TACE and ADAM10 (FIG. 7). The HPLC/HR-MS data indicate that human TACE cleaves the stalk region of human TREM2 isoform 1 between Histidine 157 and Serine 158 of SEQ ID NO: 1 (FIG. 8). Histidine 157 and Serine 158 are conserved in human TREM2 isoforms 2 and 3 (FIGS. 1B and 1C), suggesting TACE cleaves these human TREM2 isoforms at the same site.

Molecules that Stabilize TREM2

Provided herein are molecules that stabilize TREM2 (e.g., human TREM2 protein) on the cell surface. Those molecules can achieve stabilization of TREM2 (e.g., human TREM2 protein) by interfering with the proteolytic cleavage of TREM2 and/or reducing shedding of the ectodomain of the TREM2 protein. In some embodiments, those molecules specifically bind to the extracellular domain of human TREM2, e.g., the amino acid residues 14 to 174 of SEQ ID NO: 1, the amino acid residues 14 to 168 of SEQ ID NO: 3, or the amino acid residues 14 to 171 of SEQ ID NO: 4. In some embodiments, those molecules specifically bind to a stalk region of human TREM2, e.g., having an amino acid sequence of any of SEQ ID NO: 7, 8, or 9.

In some embodiments, those TREM2-binding molecules (e.g., human TREM2-binding molecules) are polypeptides, e.g., antibodies or antigen-binding fragments thereof, e.g., monoclonal antibodies. In some embodiments, those TREM2-binding molecules (e.g., human TREM2-binding molecules) are aptamers, e.g., slow off-rate modified aptamers. In some embodiments, those TREM2-binding molecules (e.g., human TREM2-binding molecules) are low molecular weight compounds.

Since dysfunctional TREM2 or absent surface TREM2 was associated with human neuroinflammatory and neurodegenerative pathologies, the TREM2-stabilizing molecules (e.g., human TREM2-binding molecules) described herein can be used to treat, prevent, or diagnose neuroinflammatory and neurodegenerative diseases such as Alzheimer's disease, frontotemporal dementia, Parkinson's disease, amyotrophic lateral sclerosis, Nasu-Hakola disease, multiple sclerosis, amyotrophic lateral sclerosis (ALS), anti-NMDA receptor encephalitis, autism, brain lupus (NP-SLE), chemo-induced peripheral neuropathy (CIPN), postherapeutic neuralgia, chronic inflammatory demyelinating polyneuropathy (CIDP), epilepsy, Guillain-Barré Syndrom (GBS), inclusion body myositis, lysosomal storage diseases, e.g., sphingomyelinlipidose (Niemann-Pick C) and mucopolysaccharidose II/IIIB, metachromatic leukodystrophy, multifocal motor neuropathy, Myasthenia Gravis, Neuro-Behcet's Disease, neuromyelitis optica (NMO), optic neuritis, polymyositis, dermatomyositis, Rasmussen's encephalitis, Rett's Syndrome, stroke, transverse myelitis, traumatic brain injury, spinal cord injury, viral encephalitis, or bacterial meningitis.

Due to their pharmacological profiles, the TREM2-binding molecules (e.g., human TREM2-binding molecules) described herein will be useful for the treatment of diseases or conditions as diverse as CNS related diseases, PNS related diseases, systemic inflammation and other diseases related to inflammation, pain and withdrawal symptoms caused by an abuse of chemical substances, diseases or disorders related to the CNS include general anxiety disorders, cognitive disorders, learning and memory deficits and dysfunctions, Alzheimer's disease (mild, moderate and severe), attention deficit and hyperactivity disorder, Parkinson's disease, dementia in Parkinson's disease, Huntington's disease, ALS, prionic neurodegenerative disorders such as Creutzfeld-Jacob disease and kuru disease, Gilles de la Tourette's syndrome, psychosis, depression and depressive disorders, mania, manic depression, schizophrenia, the cognitive deficits in schizophrenia, obsessive compulsive disorders, panic disorders, eating disorders, narcolepsy, nociception, AIDS-dementia, senile dementia, mild cognitive impairment related to age (MCI), age associated memory impairment, autism, dyslexia, tardive dyskinesia, epilepsy, and convulsive disorders, post-traumatic stress disorders, transient anoxia, pseudodementia, pre-menstrual syndrome, late luteal phase syndrome, chronic fatigue syndrome and jet lag.

The TREM2-binding molecules (e.g., human TREM2-binding molecules) are particularly suitable for treating, preventing or diagnosing autoimmune, inflammatory, or malignant disorders mediated by or associated with extensive proteolytic cleavage of TREM2 (e.g., human TREM2) or cells expressing aberrant or mutated variants of the TREM2 receptor (e.g., human TREM2 receptor). Examples of autoimmune diseases include, without limitation, arthritis (for example rheumatoid arthritis, arthritis chronic a progrediente and arthritis deformans) and rheumatic diseases, including inflammatory conditions and rheumatic diseases involving bone loss, inflammatory pain, spondyloarhropathies including ankolsing spondylitis, Reiter syndrome, reactive arthritis, psoriatic arthritis, and enterophathis arthritis, hypersensitivity (including both airways hypersensitivity and dermal hypersensitivity) and allergies. Autoimmune diseases include autoimmune haematological disorders (including e.g. hemolytic anaemia, aplastic anaemia, pure red cell anaemia and idiopathic thrombocytopenia), systemic lupus erythematosus, inflammatory muscle disorders, polychondritis, sclerodoma, Wegener granulomatosis, dermatomyositis, chronic active hepatitis, myasthenia gravis, psoriasis, Steven-Johnson syndrome, idiopathic sprue, endocrine ophthalmopathy, Graves disease, sarcoidosis, multiple sclerosis, primary biliary cirrhosis, juvenile diabetes (diabetes mellitus type I), uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including gout, langerhans cell histiocytosis, idiopathic nephrotic syndrome or minimal change nephropathy), tumors, inflammatory disease of skin and cornea, myositis, loosening of bone implants, metabolic disorders, such as atherosclerosis, diabetes, and dislipidemia.

The TREM2-binding molecules (e.g., human TREM2-binding molecules) are also useful for the treatment, prevention, or amelioration of asthma, bronchitis, pneumoconiosis, pulmonary emphysema, and other obstructive or inflammatory diseases of the airways including idiopathic pulmonary fibrosis or COPD.

The TREM2-binding molecules (e.g., human TREM2-binding molecules) can be used to treat hematopoietic or hepatopoetic malignant disorder such as acute myeloid leukemia, chronic myeloid leukemia, myeloproliferative disorders, myelodysplastic syndromes, multiple myeloma, paroxysmal nocturnal hemoglobinuria, fanconi anemi, thalassemia major, Wiskott-Aldrich syndrome, hemophagocytic lymphohistiocytosis.

The TREM2-binding molecules (e.g., human TREM2-binding molecules) can be used to treat any disease or disorder directly or indirectly associated with aberrant TREM2 activity and/or expression. The Trem2-related disorders (e.g., human TREM2-related disorders) include immunological disorders, especially involving inflammatory disorders (e.g., bacterial infection, fungal infection, viral infection, protozoa or other parasitic infection, psoriasis, septicemia, cerebral malaria, inflammatory bowel disease, arthritis, such as rheumatoid arthritis, folliculitis, impetigo, granulomas, lipoid pneumonias, vasculitis, and osteoarthritis), autoimmune disorders (e.g., rheumatoid arthritis, thyroiditis, such as Hashimoto's thyroiditis and Graves' disease, insulin-resistant diabetes, pernicious anemia, Addison's disease, pemphigus, vitiligo, ulcerative colitis, systemic lupus erythematosus (SLE), Sjogren's syndrome, multiple sclerosis, dermatomyositis, mixed connective tissue disease, scleroderma, polymyositis, graft rejection, such as allograft rejection), T cell disorders (e.g., AIDS), allergic inflammatory disorders (e.g., skin and/or mucosal allergies, such as allergic rhinitis, asthma, psoriasis), neurological disorders, eye disorders, embryonic disorders, or any other disorders (e.g., tumors, cancers, leukemia, myeloid diseases, and traumas) which are directly or indirectly associated with aberrant TREM2 activity and/or expression.

In some embodiments, the Trem2-related disorder (e.g., human TREM2-related disorder) is selected from asthma, encephalitis, inflammatory bowel disease, chronic obstructive pulmonary disease (COPD), allergic disorders, septic shock, pulmonary fibrosis, undifferentiated spondyloarthropathy, undifferentiated arthropathy, arthritis, inflammatory osteolysis, or chronic inflammation resulting from chronic viral or bacterial infections.

In some embodiments, the Trem2-related disorder (e.g., human TREM2-related disorder) is selected from dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, Taupathy disease, Nasu-Hakola disease, stroke, acute trauma, chronic trauma, lupus, acute and chronic colitis, wound healing, Crohn's disease, inflammatory bowel disease, ulcerative colitis, obesity, Malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, Sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, and cancer.

In some embodiments, the Trem2-related disorder (e.g., human TREM2-related disorder) is selected from dementia, frontotemporal dementia, Alzheimer's disease, Nasu-Hakola disease, and multiple sclerosis. In some embodiments, Trem2-related disorder (e.g., human TREM2-related disorder) is a dementia such as frontotemporal dementia, Alzheimer's disease, vascular dementia, semantic dementia, or dementia with Lewy bodies.

Antibodies

Provided herein are antibodies or antigen-binding fragments thereof that specifically bind to the extracellular domain of TREM2 protein (e.g., human TREM2 protein) and stabilize the TREM2 protein (e.g., human TREM2 protein). In some embodiments, those antibodies or antigen-binding fragments thereof specifically bind to the extracellular domain of human TREM2, e.g., the amino acid residues 14 to 174 of SEQ ID NO: 1, the amino acid residues 14 to 168 of SEQ ID NO: 3, or the amino acid residues 14 to 171 of SEQ ID NO: 4.

In some embodiments, those antibodies or antigen-binding fragments thereof specifically bind to a stalk region of human TREM2, e.g., having an amino acid sequence of any of SEQ ID NO: 7, 8, or 9. Those antibodies or antigen-binding fragments can stabilize the human TREM2 protein on the cell surface, and/or reduce shedding of the ectodomain of the human TREM2 protein.

In some embodiments, an antibody of the invention has a full length antibody heavy chain sequence and a full length antibody light chain sequence. In some embodiments, an antibody of the invention has a heavy chain variable region (V_{H}) and a light chain variable region (V_{L}). In some embodiments, an antibody of the invention has a heavy chain variable region comprising CDR1, CDR2, and CDR3 sequences and a light chain variable region comprising CDR1, CDR2, and CDR3 sequences.

In some embodiments, the present invention provides an antibody or antigen-binding fragment thereof, which bind to the extracellular domain of human TREM2 protein with a dissociation constant (K_{D}) of less than 100 pM, e.g., a K_{D} of less than 90 pM, less than 80 pM, less than 70 pM, less than 60 pM, less than 50 pM, less than 40 pM, less than 30 pM, less than 20 pM, less than 10 pM. In some embodiments, the antibodies or antigen-binding fragments provided herein bind to the extracellular domain of human TREM2 protein with a dissociation constant (K_{D}) of less than 10 pM.

In some embodiments, the present invention provides an antibody or antigen-binding fragment thereof, which bind to a stalk region of human TREM2, e.g., having an amino acid sequence of any of SEQ ID NO: 7, 8, or 9, with a dissociation constant (K_{D}) of less than 100 pM, e.g., a K_{D} of less than 90 pM, less than 80 pM, less than 70 pM, less than 60 pM, less than 50 pM, less than 40 pM, less than 30 pM, less than 20 pM, less than 10 pM. In some embodiments, the antibodies or antigen-binding fragments provided herein bind to a stalk region of human TREM2, e.g., having an amino acid sequence of any of SEQ ID NO: 7, 8, or 9, with a dissociation constant (K_{D}) of less than 10 pM.

Once a desired epitope on an antigen is determined, it is possible to generate antibodies to that epitope, e.g., using the techniques described in the present invention. Alternatively, during the discovery process, the generation and characterization of antibodies may elucidate information about desirable epitopes. From this information, it is then possible to competitively screen antibodies for binding to the same epitope. An approach to achieve this is to conduct cross-competition studies to find antibodies that competitively bind with one another, e.g., the antibodies compete for binding to the antigen. A high throughput process for “binning” antibodies based upon their cross-competition is described in International Patent Application No. WO 2003/48731. As will be appreciated by one of skill in the art, practically anything to which an antibody can specifically bind could be an epitope. An epitope can comprises those residues to which the antibody binds.

Generally, antibodies specific for a particular target antigen will preferentially recognize an epitope on the target antigen in a complex mixture of proteins and/or macromolecules.

Regions of a given polypeptide that include an epitope can be identified using any number of epitope mapping techniques, well known in the art. See, e.g., Epitope Mapping Protocols in Methods in Molecular Biology, Vol. 66 (Glenn E. Morris, Ed., 1996, Humana Press, Totowa, N.J). For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al., (1984) Proc. Natl. Acad. Sci. USA 8:3998-4002; Geysen et al., (1985) Proc. Natl. Acad. Sci. USA 82:78-182; Geysen et al., (1986) Mol. Immunol. 23:709-715. Similarly, conformational epitopes are readily identified by determining spatial conformation of amino acids such as by, e.g., x-ray crystallography and two-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al., (1981) Proc. Natl. Acad. Sci USA 78:3824-3828; for determining antigenicity profiles, and the Kyte-Doolittle technique, Kyte et al., (1982) J. Mol. Biol. 157:105-132; for hydropathy plots.

In some embodiments, an anti-TREM2 antibody specifically binds to an epitope in the extracellular domain of human TREM2. For example, an anti-TREM2 antibody can specifically bind to an epitope within the amino acid residues 14 to 174 of SEQ ID NO: 1, the amino acid residues 14 to 168 of SEQ ID NO: 3, or the amino acid residues 14 to 171 of SEQ ID NO: 4. In some embodiments, an anti-TREM2 antibody specifically binds to an epitope in the stalk region of human TREM2. For example, an anti-TREM2 antibody can specifically bind to an epitope within any of SEQ ID NO: 7, 8, or 9. In some embodiments, an anti-TREM2 antibody specifically binds to an epitope in the IgV domain of human TREM2. For example, an anti-TREM2 antibody can specifically bind to an epitope within the amino acid residues 14 to 112 of SEQ ID NO: 1.

Chimeric and/or humanized antibodies, can be engineered to minimize the immune response by a human patient to antibodies produced in non-human subjects or derived from the expression of non-human antibody genes. Chimeric antibodies comprise a non-human animal antibody variable region and a human antibody constant region. Such antibodies retain the epitope binding specificity of the original monoclonal antibody, but may be less immunogenic when administered to humans, and therefore more likely to be tolerated by the patient. For example, one or all (e.g., one, two, or three) of the variable regions of the light chain(s) and/or one or all (e.g., one, two, or three) of the variable regions the heavy chain(s) of a mouse antibody (e.g., a mouse monoclonal antibody) can each be joined to a human constant region, such as, without limitation an IgG1 human constant region. Chimeric monoclonal antibodies can be produced by recombinant DNA techniques known in the art. For example, a gene encoding the constant region of a non-human antibody molecule can be substituted with a gene encoding a human constant region (see Robinson et al., PCT Patent Publication PCT/US86/02269; Akira, et al., European Patent Application 184,187; or Taniguchi, M., European Patent Application 171,496). In addition, other suitable techniques that can be used to generate chimeric antibodies are described, for example, in U.S. Pat. Nos. 4,816,567; 4,978,775; 4,975,369; and 4,816,397.

A chimeric antibody can be further “humanized” by replacing portions of the variable region not involved in antigen binding with equivalent portions from human variable regions. Humanized antibodies comprise one or more human framework regions in the variable region together with non-human (e.g., mouse, rat, or hamster) complementarity-determining regions (CDRs) of the heavy and/or light chain. In some embodiments, a humanized antibody comprises sequences that are entirely human except for the CDR regions. Humanized antibodies are typically less immunogenic to humans, relative to non-humanized antibodies, and thus offer therapeutic benefits in certain situations. Humanized TREM2 antibodies can be generated using methods known in the art. See for example, Hwang et al., Methods 36:35, 2005; Queen et al., Proc. Natl. Acad. Sci. U.S.A. 86:10029-10033, 1989; Jones et al., Nature 321:522-25, 1986; Riechmann et al., Nature 332:323-27, 1988; Verhoeyen et al., Science 239:1534-36, 1988; Orlandi et al., Proc. Natl. Acad. Sci. U.S.A. 86:3833-3837, 1989; U.S. Pat. Nos. 5,225,539; 5,530,101; 5,585,089; 5,693,761; 5,693,762; and 6,180,370; and WO 90/07861.

Human TREM2 antibodies can be generated using methods that are known in the art. For example, the humaneering technology used to converting non-human antibodies into engineered human antibodies. U.S. Patent Publication No. 20050008625 describes an in vivo method for replacing a nonhuman antibody variable region with a human variable region in an antibody while maintaining the same or providing better binding characteristics relative to that of the nonhuman antibody. The method relies on epitope guided replacement of variable regions of a non-human reference antibody with a fully human antibody. The resulting human antibody is generally structurally unrelated to the reference nonhuman antibody, but binds to the same epitope on the same antigen as the reference antibody. Briefly, the serial epitope-guided complementarity replacement approach is enabled by setting up a competition in cells between a “competitor” and a library of diverse hybrids of the reference antibody (“test antibodies”) for binding to limiting amounts of antigen in the presence of a reporter system which responds to the binding of test antibody to antigen. The competitor can be the reference antibody or derivative thereof such as a single-chain Fv fragment. The competitor can also be a natural or artificial ligand of the antigen which binds to the same epitope as the reference antibody. The only requirements of the competitor are that it binds to the same epitope as the reference antibody, and that it competes with the reference antibody for antigen binding. The test antibodies have one antigen-binding V-region in common from the nonhuman reference antibody, and the other V-region selected at random from a diverse source such as a repertoire library of human antibodies. The common V-region from the reference antibody serves as a guide, positioning the test antibodies on the same epitope on the antigen, and in the same orientation, so that selection is biased toward the highest antigen-binding fidelity to the reference antibody.

Many types of reporter system can be used to detect desired interactions between test antibodies and antigen. For example, complementing reporter fragments may be linked to antigen and test antibody, respectively, so that reporter activation by fragment complementation only occurs when the test antibody binds to the antigen. When the test antibody- and antigen-reporter fragment fusions are co-expressed with a competitor, reporter activation becomes dependent on the ability of the test antibody to compete with the competitor, which is proportional to the affinity of the test antibody for the antigen. Other reporter systems that can be used include the reactivator of an auto-inhibited reporter reactivation system (RAIR) as disclosed in U.S. patent application Ser. No. 10/208,730 (Publication No. 20030198971), or competitive activation system disclosed in U.S. patent application Ser. No. 10/076,845 (Publication No. 20030157579).

With the serial epitope-guided complementarity replacement system, selection is made to identify cells expresses a single test antibody along with the competitor, antigen, and reporter components. In these cells, each test antibody competes one-on-one with the competitor for binding to a limiting amount of antigen. Activity of the reporter is proportional to the amount of antigen bound to the test antibody, which in turn is proportional to the affinity of the test antibody for the antigen and the stability of the test antibody. Test antibodies are initially selected on the basis of their activity relative to that of the reference antibody when expressed as the test antibody. The result of the first round of selection is a set of “hybrid” antibodies, each of which is comprised of the same non-human V-region from the reference antibody and a human V-region from the library, and each of which binds to the same epitope on the antigen as the reference antibody. One of more of the hybrid antibodies selected in the first round will have an affinity for the antigen comparable to or higher than that of the reference antibody.

In the second V-region replacement step, the human V-regions selected in the first step are used as guide for the selection of human replacements for the remaining non-human reference antibody V-region with a diverse library of cognate human V-regions. The hybrid antibodies selected in the first round may also be used as competitors for the second round of selection. The result of the second round of selection is a set of fully human antibodies which differ structurally from the reference antibody, but which compete with the reference antibody for binding to the same antigen. Some of the selected human antibodies bind to the same epitope on the same antigen as the reference antibody. Among these selected human antibodies, one or more binds to the same epitope with an affinity which is comparable to or higher than that of the reference antibody.

Using a mouse or chimeric TREM2 antibody, human antibodies that bind to human TREM2 with the same binding specificity and the same or better binding affinity can be generated. In addition, such human TREM2 antibodies can also be commercially obtained from companies which customarily produce human antibodies, e.g., KaloBios, Inc. (Mountain View, Calif.).

Engineered and Modified Antibodies

An antibody of the invention further can be prepared using an antibody having one or more of the VH and/or VL sequences as starting material to engineer a modified antibody, which modified antibody may have altered properties from the starting antibody. An antibody can be engineered by modifying one or more residues within one or both variable regions (i.e., VH and/or VL), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al., 1998 Nature 332:323-327; Jones, P. et al., 1986 Nature 321:522-525; Queen, C. et al., 1989 Proc. Natl. Acad., U.S.A. 86:10029-10033; U.S. Pat. No. 5,225,539 to Winter, and U.S. Pat. Nos. 5,530,101; 5,585,089; 5,693,762 and 6,180,370 to Queen et al.)

Such framework sequences can be obtained from public DNA databases or published references that include germline antibody gene sequences or rearranged antibody sequences. For example, germline DNA sequences for human heavy and light chain variable region genes can be found in the “VBase” human germline sequence database (available on the Internet at www.mrc-cpe.cam.ac.uk/vbase), as well as in Kabat, E. A., et al., 1991 Sequences of Proteins of Immunological Interest, Fifth Edition, U.S. Department of Health and Human Services, NIH Publication No. 91-3242; Tomlinson, I. M., et al., 1992 J. fol. Biol. 227:776-798; and Cox, J. P. L. et al., 1994 Eur. J Immunol. 24:827-836; the contents of each of which are expressly incorporated herein by reference. For example, germline DNA sequences for human heavy and light chain variable region genes and rearranged antibody sequences can be found in “IMGT” database (available on the Internet at www.imgt.org; see Lefranc, M. P. et al., 1999 Nucleic Acids Res. 27:209-212; the contents of each of which are expressly incorporated herein by reference.)

An example of framework sequences for use in the antibodies and antigen-binding fragments thereof of the invention are those that are structurally similar to the framework sequences used by selected antibodies and antigen-binding fragments thereof of the invention, e.g., consensus sequences and/or framework sequences used by monoclonal antibodies of the invention. The VH CDR1, 2 and 3 sequences, and the VL CDR1, 2 and 3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al).

Another type of variable region modification is to mutate amino acid residues within the VH and/or VL CDR1, CDR2 and/or CDR3 regions to thereby improve one or more binding properties (e.g., affinity) of the antibody of interest, known as “affinity maturation.” Site-directed mutagenesis or PCR-mediated mutagenesis can be performed to introduce the mutation (s) and the effect on antibody binding, or other functional property of interest, can be evaluated in in vitro or in vivo assays as described herein and provided in the Examples. Conservative modifications (as discussed above) can be introduced. The mutations may be amino acid substitutions, additions or deletions. Moreover, typically no more than one, two, three, four or five residues within a CDR region are altered.

A wide variety of antibody/immunoglobulin frameworks or scaffolds can be employed so long as the resulting polypeptide includes at least one binding region which specifically binds to TREM2. Such frameworks or scaffolds include the 5 main idiotypes of human immunoglobulins, antigen-binding fragments thereof, and include immunoglobulins of other animal species, preferably having humanized aspects. Single heavy-chain antibodies such as those identified in camelids are of particular interest in this regard. Novel frameworks, scaffolds and fragments continue to be discovered and developed by those skilled in the art.

In one aspect, the invention pertains to a method of generating non-immunoglobulin based antibodies using non-immunoglobulin scaffolds onto which CDRs of the invention can be grafted. Known or future non-immunoglobulin frameworks and scaffolds may be employed, as long as they comprise a binding region specific for the target TREM2 protein. Known non-immunoglobulin frameworks or scaffolds include, but are not limited to, fibronectin (Compound Therapeutics, Inc., Waltham, Mass.), ankyrin (Molecular Partners AG, Zurich, Switzerland), domain antibodies (Domantis, Ltd., Cambridge, Mass., and Ablynx nv, Zwijnaarde, Belgium), lipocalin (Pieris Proteolab AG, Freising, Germany), small modular immuno-pharmaceuticals (Trubion Pharmaceuticals Inc., Seattle, Wash.), maxybodies (Avidia, Inc., Mountain View, Calif.), Protein A (Affibody AG, Sweden), and affilin (gamma-crystallin or ubiquitin) (Scil Proteins GmbH, Halle, Germany).

The fibronectin scaffolds are based on fibronectin type III domain (e.g., the tenth module of the fibronectin type III (10 Fn3 domain)). The fibronectin type III domain has 7 or 8 beta strands which are distributed between two beta sheets, which themselves pack against each other to form the core of the protein, and further containing loops (analogous to CDRs) which connect the beta strands to each other and are solvent exposed. There are at least three such loops at each edge of the beta sheet sandwich, where the edge is the boundary of the protein perpendicular to the direction of the beta strands (see U.S. Pat. No. 6,818,418). These fibronectin-based scaffolds are not an immunoglobulin, although the overall fold is closely related to that of the smallest functional antibody fragment, the variable region of the heavy chain, which comprises the entire antigen recognition unit in camel and llama IgG. Because of this structure, the non-immunoglobulin antibody mimics antigen binding properties that are similar in nature and affinity for those of antibodies. These scaffolds can be used in a loop randomization and shuffling strategy in vitro that is similar to the process of affinity maturation of antibodies in vivo. These fibronectin-based molecules can be used as scaffolds where the loop regions of the molecule can be replaced with CDRs of the invention using standard cloning techniques.

The ankyrin technology is based on using proteins with ankyrin derived repeat modules as scaffolds for bearing variable regions which can be used for binding to different targets. The ankyrin repeat module is a 33 amino acid polypeptide consisting of two anti-parallel alpha-helices and a beta-turn. Binding of the variable regions is mostly optimized by using ribosome display.

Avimers are derived from natural A-domain containing protein such as LRP-1. These domains are used by nature for protein-protein interactions and in human over 250 proteins are structurally based on A-domains. Avimers consist of a number of different “A-domain” monomers (2-10) linked via amino acid linkers. Avimers can be created that can bind to the target antigen using the methodology described in, for example, U.S. Patent Application Publication Nos. 20040175756; 20050053973; 20050048512; and 20060008844.

Affibody affinity ligands are small, simple proteins composed of a three-helix bundle based on the scaffold of one of the IgG-binding domains of Protein A. Protein A is a surface protein from the bacterium Staphylococcus aureus. This scaffold domain consists of 58 amino acids, 13 of which are randomized to generate affibody libraries with a large number of ligand variants (See e.g., U.S. Pat. No. 5,831,012). Affibody molecules mimic antibodies, they have a molecular weight of 6 kDa, compared to the molecular weight of antibodies, which is 150 kDa. In spite of its small size, the binding site of affibody molecules is similar to that of an antibody.

Anticalins are products developed by the company Pieris ProteoLab AG. They are derived from lipocalins, a widespread group of small and robust proteins that are usually involved in the physiological transport or storage of chemically sensitive or insoluble compounds. Several natural lipocalins occur in human tissues or body liquids. The protein architecture is reminiscent of immunoglobulins, with hypervariable loops on top of a rigid framework. However, in contrast with antibodies or their recombinant fragments, lipocalins are composed of a single polypeptide chain with 160 to 180 amino acid residues, being just marginally bigger than a single immunoglobulin domain. The set of four loops, which makes up the binding pocket, shows pronounced structural plasticity and tolerates a variety of side chains. The binding site can thus be reshaped in a proprietary process in order to recognize prescribed target molecules of different shape with high affinity and specificity. One protein of lipocalin family, the bilin-binding protein (BBP) of Pieris Brassicae has been used to develop anticalins by mutagenizing the set of four loops. One example of a patent application describing anticalins is in PCT Publication No. WO 199916873.

Affilin molecules are small non-immunoglobulin proteins which are designed for specific affinities towards proteins and small molecules. New affilin molecules can be very quickly selected from two libraries, each of which is based on a different human derived scaffold protein. Affilin molecules do not show any structural homology to immunoglobulin proteins. Currently, two affilin scaffolds are employed, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of gamma crystalline derived proteins are described in WO200104144 and examples of “ubiquitin-like” proteins are described in WO2004106368.

Protein epitope mimetics (PEM) are medium-sized, cyclic, peptide-like molecules (MW 1-2 kDa) mimicking beta-hairpin secondary structures of proteins, the major secondary structure involved in protein-protein interactions.

The human TREM2-binding antibodies can be generated using methods that are known in the art. For example, the humaneering technology used to converting non-human antibodies into engineered human antibodies. U.S. Patent Publication No. 20050008625 describes an in vivo method for replacing a nonhuman antibody variable region with a human variable region in an antibody while maintaining the same or providing better binding characteristics relative to that of the nonhuman antibody. The method relies on epitope guided replacement of variable regions of a non-human reference antibody with a fully human antibody. The resulting human antibody is generally unrelated structurally to the reference nonhuman antibody, but binds to the same epitope on the same antigen as the reference antibody. Briefly, the serial epitope-guided complementarity replacement approach is enabled by setting up a competition in cells between a “competitor” and a library of diverse hybrids of the reference antibody (“test antibodies”) for binding to limiting amounts of antigen in the presence of a reporter system which responds to the binding of test antibody to antigen. The competitor can be the reference antibody or derivative thereof such as a single-chain Fv fragment. The competitor can also be a natural or artificial ligand of the antigen which binds to the same epitope as the reference antibody. The only requirements of the competitor are that it binds to the same epitope as the reference antibody, and that it competes with the reference antibody for antigen binding. The test antibodies have one antigen-binding V-region in common from the nonhuman reference antibody, and the other V-region selected at random from a diverse source such as a repertoire library of human antibodies. The common V-region from the reference antibody serves as a guide, positioning the test antibodies on the same epitope on the antigen, and in the same orientation, so that selection is biased toward the highest antigen-binding fidelity to the reference antibody.

Many types of reporter system can be used to detect desired interactions between test antibodies and antigen. For example, complementing reporter fragments may be linked to antigen and test antibody, respectively, so that reporter activation by fragment complementation only occurs when the test antibody binds to the antigen. When the test antibody- and antigen-reporter fragment fusions are co-expressed with a competitor, reporter activation becomes dependent on the ability of the test antibody to compete with the competitor, which is proportional to the affinity of the test antibody for the antigen. Other reporter systems that can be used include the reactivator of an auto-inhibited reporter reactivation system (RAIR) as disclosed in U.S. patent application Ser. No. 10/208,730 (Publication No. 20030198971), or competitive activation system disclosed in U.S. patent application Ser. No. 10/076,845 (Publication No. 20030157579).

With the serial epitope-guided complementarity replacement system, selection is made to identify cells expresses a single test antibody along with the competitor, antigen, and reporter components. In these cells, each test antibody competes one-on-one with the competitor for binding to a limiting amount of antigen. Activity of the reporter is proportional to the amount of antigen bound to the test antibody, which in turn is proportional to the affinity of the test antibody for the antigen and the stability of the test antibody. Test antibodies are initially selected on the basis of their activity relative to that of the reference antibody when expressed as the test antibody. The result of the first round of selection is a set of “hybrid” antibodies, each of which is comprised of the same non-human V-region from the reference antibody and a human V-region from the library, and each of which binds to the same epitope on the antigen as the reference antibody. One of more of the hybrid antibodies selected in the first round will have an affinity for the antigen comparable to or higher than that of the reference antibody.

In the second V-region replacement step, the human V-regions selected in the first step are used as guide for the selection of human replacements for the remaining non-human reference antibody V-region with a diverse library of cognate human V-regions. The hybrid antibodies selected in the first round may also be used as competitors for the second round of selection. The result of the second round of selection is a set of fully human antibodies which differ structurally from the reference antibody, but which compete with the reference antibody for binding to the same antigen. Some of the selected human antibodies bind to the same epitope on the same antigen as the reference antibody. Among these selected human antibodies, one or more binds to the same epitope with an affinity which is comparable to or higher than that of the reference antibody.

Camelid Antibodies

Antibody proteins obtained from members of the camel and dromedary (Camelus bactrianus and Calelus dromaderius) family including new world members such as llama species (Lama paccos, Lama glama and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals. See PCT/EP93/02214 (WO 94/04678 published 3 Mar. 1994).

A region of the camelid antibody which is the small single variable domain identified as VHH can be obtained by genetic engineering to yield a small protein having high affinity for a target, resulting in a low molecular weight antibody-derived protein known as a “camelid nanobody.” See U.S. Pat. No. 5,759,808 issued Jun. 2, 1998; see also Stijlemans, B. et al., 2004 J Biol Chem 279: 1256-1261; Dumoulin, M. et al., 2003 Nature 424: 783-788; Pleschberger, M. et al. 2003 Bioconjugate Chem 14: 440-448; Cortez-Retamozo, V. et al. 2002 Int J Cancer 89: 456-62; and Lauwereys, M. et al. 1998 EMBO J 17: 3512-3520. Engineered libraries of camelid antibodies and antibody fragments are commercially available, for example, from Ablynx, Ghent, Belgium. As with other antibodies and antigen-binding fragments thereof of non-human origin, an amino acid sequence of a camelid antibody can be altered recombinantly to obtain a sequence that more closely resembles a human sequence, i.e., the nanobody can be “humanized” Thus the natural low antigenicity of camelid antibodies to humans can be further reduced.

The camelid nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins, i.e., camelid nanobodies are useful as reagents detect antigens that are otherwise cryptic using classical immunological techniques, and as possible therapeutic agents. Thus yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody.

The low molecular weight and compact size further result in camelid nanobodies being extremely thermostable, stable to extreme pH and to proteolytic digestion, and poorly antigenic. Another consequence is that camelid nanobodies readily move from the circulatory system into tissues, and even cross the blood-brain barrier and can treat disorders that affect nervous tissue. Nanobodies can further facilitated drug transport across the blood brain barrier. See U.S. patent application 20040161738 published Aug. 19, 2004. These features combined with the low antigenicity to humans indicate great therapeutic potential. Further, these molecules can be fully expressed in prokaryotic cells such as E. coli and are expressed as fusion proteins with bacteriophage and are functional.

Accordingly, a feature of the present invention is a camelid antibody or nanobody having high affinity for TREM2. In one embodiment herein, the camelid antibody or nanobody is naturally produced in the camelid animal, i.e., is produced by the camelid following immunization with TREM2 or a peptide fragment thereof, using techniques described herein for other antibodies. Alternatively, the TREM2-binding camelid nanobody is engineered, i.e., produced by selection for example from a library of phage displaying appropriately mutagenized camelid nanobody proteins using panning procedures with TREM2 as a target as described in the examples herein. Engineered nanobodies can further be customized by genetic engineering to have a half life in a recipient subject of from 45 minutes to two weeks. In a specific embodiment, the camelid antibody or nanobody is obtained by grafting the CDRs sequences of the heavy or light chain of the human antibodies of the invention into nanobody or single domain antibody framework sequences, as described for example in PCT/EP93/02214.

Bispecific Molecules and Multivalent Antibodies

In another aspect, the present invention features bispecific or multispecific molecules comprising a TREM2-binding antibody, or a fragment thereof, of the invention. An antibody of the invention, or antigen-binding regions thereof, can be derivatized or linked to another functional molecule, e.g., another peptide or protein (e.g., another antibody or ligand for a receptor) to generate a bispecific molecule that binds to at least two different binding sites or target molecules. The antibody of the invention may in fact be derivatized or linked to more than one other functional molecule to generate multi-specific molecules that bind to more than two different binding sites and/or target molecules; such multi-specific molecules are also intended to be encompassed by the term “bispecific molecule” as used herein. To create a bispecific molecule of the invention, an antibody of the invention can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, the present invention includes bispecific molecules comprising at least one first binding specificity for TREM2 and a second binding specificity for a second target epitope. For example, the second target epitope is another epitope of TREM2 different from the first target epitope.

Additionally, for the invention in which the bispecific molecule is multi-specific, the molecule can further include a third binding specificity, in addition to the first and second target epitope.

In one embodiment, the bispecific molecules of the invention comprise as a binding specificity at least one antibody, or an antibody fragment thereof, including, e.g., a Fab, Fab′, F (ab′)2, Fv, or a single chain Fv. The antibody may also be a light chain or heavy chain dimer, or any minimal fragment thereof such as a Fv or a single chain construct as described in Ladner et al. U.S. Pat. No. 4,946,778.

Diabodies are bivalent, bispecific molecules in which VH and VL domains are expressed on a single polypeptide chain, connected by a linker that is too short to allow for pairing between the two domains on the same chain. The VH and VL domains pair with complementary domains of another chain, thereby creating two antigen binding sites (see e.g., Holliger et al., 1993 Proc. Natl. Acad. Sci. USA 90:6444-6448; Poijak et al., 1994 Structure 2:1121-1123). Diabodies can be produced by expressing two polypeptide chains with either the structure VHA-VLB and VHB-VLA (VH-VL configuration), or VLA-VHB and VLB-VHA (VL-VH configuration) within the same cell. Most of them can be expressed in soluble form in bacteria. Single chain diabodies (scDb) are produced by connecting the two diabody-forming polypeptide chains with linker of approximately 15 amino acid residues (see Holliger and Winter, 1997 Cancer Immunol. Immunother., 45 (3-4):128-30; Wu et al., 1996 Immunotechnology, 2 (1):21-36). scDb can be expressed in bacteria in soluble, active monomeric form (see Holliger and Winter, 1997 Cancer Immunol. Immunother., 45 (34): 128-30; Wu et al., 1996 Immunotechnology, 2 (1):21-36; Pluckthun and Pack, 1997 Immunotechnology, 3 (2): 83-105; Ridgway et al., 1996 Protein Eng., 9 (7):617-21). A diabody can be fused to Fc to generate a “di-diabody” (see Lu et al., 2004 J. Biol. Chem., 279 (4):2856-65).

Other antibodies which can be employed in the bispecific molecules of the invention are murine, chimeric and humanized monoclonal antibodies.

The bispecific molecules of the present invention can be prepared by conjugating the constituent binding specificities, using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-5-acetyl-thioacetate (SATA), 5,5′-dithiobis (2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohaxane-1-carboxylate (sulfo-SMCC) (see e.g., Karpovsky et al., 1984 J. Exp. Med. 160:1686; Liu, M A et al., 1985 Proc. Natl. Acad. Sci. USA 82:8648). Other methods include those described in Paulus, 1985 Behring Ins. Mitt. No. 78, 118-132; Brennan et al., 1985 Science 229:81-83), and Glennie et al., 1987 J. Immunol. 139: 2367-2375). Conjugating agents are SATA and sulfo-SMCC, both available from Pierce Chemical Co. (Rockford, Ill.).

When the binding specificities are antibodies, they can be conjugated by sulfhydryl bonding of the C-terminus hinge regions of the two heavy chains. In a particularly embodiment, the hinge region is modified to contain an odd number of sulfhydryl residues, for example one, prior to conjugation.

Alternatively, both binding specificities can be encoded in the same vector and expressed and assembled in the same host cell. This method is particularly useful where the bispecific molecule is a mAb X mAb, mAb X Fab, Fab X F (ab′)2 or ligand X Fab fusion protein. A bispecific molecule of the invention can be a single chain molecule comprising one single chain antibody and a binding determinant, or a single chain bispecific molecule comprising two binding determinants Bispecific molecules may comprise at least two single chain molecules. Methods for preparing bispecific molecules are described for example in U.S. Pat. Nos. 5,260,203; 5,455,030; 4,881,175; 5,132,405; 5,091,513; 5,476,786; 5,013,653; 5,258,498; and 5,482,858.

Binding of the bispecific molecules to their specific targets can be confirmed by, for example, enzyme-linked immunosorbent assay (ELISA), radioimmunoassay (REA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest.

In another aspect, the present invention provides multivalent compounds comprising at least two identical or different antigen-binding portions of the antibodies and antigen-binding fragments thereof of the invention binding to TREM2. The antigen-binding portions can be linked together via protein fusion or covalent or noncovalent linkage. Alternatively, methods of linkage has been described for the bispecific molecules. Tetravalent compounds can be obtained for example by cross-linking antibodies and antigen-binding fragments thereof of the invention with an antibody or antigen-binding fragment that binds to the constant regions of the antibodies and antigen-binding fragments thereof of the invention, for example the Fc or hinge region.

Trimerizing domain are described for example in Borean patent EP 1 012 280B1. Pentamerizing modules are described for example in PCT/EP97/05897.

In some embodiments, the TREM2-binding molecule is a bispecific antibody that binds to both TREM2 (e.g., human TREM2 protein) and DAP12 (e.g., human DAP12 protein). In some embodiments, the TREM2-binding molecule is a bispecific antibody that recognizes a first antigen and a second antigen. In some embodiments, the first antigen is human TREM2 or a naturally occurring variant thereof. In some embodiments, the second antigen is human DAP12 or a naturally occurring variant thereof. In some embodiments, the second antigen is human DAP10 or a Siglec (Sialic acid-binding immunoglobulin-type lectin). In some embodiments, the second antigen is a disease-causing protein selected from amyloid beta or fragments thereof, Tau, IAPP, alpha-synuclein, TDP-43, FUS protein, prion protein, PrPSc, huntingtin, calcitonin, superoxide dismutase, ataxin, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, and proline-arginine (PR) repeat peptides. In some embodiments, the second antigen is a blood brain barrier targeting protein selected from transferin receptor, insulin receptor, insulin like growth factor receptor, LRP-1, and LRP1; or ligands and/or proteins expressed on immune cells, wherein the ligands and/or proteins selected from the group consisting of: CD40, OX40, ICOS, CD28, CD137/4-1BB, CD27, GITR, PD-L1, CTLA4, PD-L2, PD-1, B7-H3, B7-H4, HVEM, BTLA, KIR, GALS, TIM3, A2AR, LAG, and phosphatidylserine. Alternatively, the second antigen may be a protein expressed on one or more tumor cells.

Antibodies with Extended Half Life

The present invention provides for antibodies that specifically bind to TREM2 (e.g., human TREM2 protein) and have an extended half-life in vivo.

Many factors may affect a protein's half life in vivo. For examples, kidney filtration, metabolism in the liver, degradation by proteolytic enzymes (proteases), and immunogenic responses (e.g., protein neutralization by antibodies and uptake by macrophages and dendritic cells). A variety of strategies can be used to extend the half life of the antibodies and antigen-binding fragments thereof of the present invention. For example, by chemical linkage to polyethylene glycol (PEG), reCODE PEG, antibody scaffold, polysialic acid (PSA), hydroxyethyl starch (HES), albumin-binding ligands, and carbohydrate shields; by genetic fusion to proteins binding to serum proteins, such as albumin, IgG, FcRn, and transferring; by coupling (genetically or chemically) to other binding moieties that bind to serum proteins, such as nanobodies, Fabs, DARPins, avimers, affibodies, and anticalins; by genetic fusion to rPEG, albumin, domain of albumin, albumin-binding proteins, and Fc; or by incorporation into nancarriers, slow release formulations, or medical devices.

To prolong the serum circulation of antibodies in vivo, inert polymer molecules such as high molecular weight PEG can be attached to the antibodies or a fragment thereof with or without a multifunctional linker either through site-specific conjugation of the PEG to the N- or C-terminus of the antibodies or via epsilon-amino groups present on lysine residues. To pegylate an antibody, the antibody, antigen-binding fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10)alkoxy- or aryloxy-polyethylene glycol or polyethylene glycol-maleimide. In one embodiment, the antibody to be pegylated is an aglycosylated antibody. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods well-known to those of skill in the art, for example, by immunoassays described herein. Methods for pegylating proteins are known in the art and can be applied to the antibodies and antigen-binding fragments thereof of the invention. See for example, EP 0154316 by Nishimura et al. and EP 0401384 by Ishikawa et al.

Other modified pegylation technologies include reconstituting chemically orthogonal directed engineering technology (ReCODE PEG), which incorporates chemically specified side chains into biosynthetic proteins via a reconstituted system that includes tRNA synthetase and tRNA. This technology enables incorporation of more than 30 new amino acids into biosynthetic proteins in E. coli, yeast, and mammalian cells. The tRNA incorporates a normative amino acid any place an amber codon is positioned, converting the amber from a stop codon to one that signals incorporation of the chemically specified amino acid.

Recombinant pegylation technology (rPEG) can also be used for serum halflife extension. This technology involves genetically fusing a 300-600 amino acid unstructured protein tail to an existing pharmaceutical protein. Because the apparent molecular weight of such an unstructured protein chain is about 15-fold larger than its actual molecular weight, the serum halflife of the protein is greatly increased. In contrast to traditional PEGylation, which requires chemical conjugation and repurification, the manufacturing process is greatly simplified and the product is homogeneous.

Polysialytion is another technology, which uses the natural polymer polysialic acid (PSA) to prolong the active life and improve the stability of therapeutic peptides and proteins. PSA is a polymer of sialic acid (a sugar). When used for protein and therapeutic peptide drug delivery, polysialic acid provides a protective microenvironment on conjugation. This increases the active life of the therapeutic protein in the circulation and prevents it from being recognized by the immune system. The PSA polymer is naturally found in the human body. It was adopted by certain bacteria which evolved over millions of years to coat their walls with it. These naturally polysialylated bacteria were then able, by virtue of molecular mimicry, to foil the body's defense system. PSA, nature's ultimate stealth technology, can be easily produced from such bacteria in large quantities and with predetermined physical characteristics. Bacterial PSA is completely non-immunogenic, even when coupled to proteins, as it is chemically identical to PSA in the human body.

Another technology include the use of hydroxyethyl starch (“HES”) derivatives linked to antibodies. HES is a modified natural polymer derived from waxy maize starch and can be metabolized by the body's enzymes. HES solutions are usually administered to substitute deficient blood volume and to improve the rheological properties of the blood. Hesylation of an antibody enables the prolongation of the circulation half-life by increasing the stability of the molecule, as well as by reducing renal clearance, resulting in an increased biological activity. By varying different parameters, such as the molecular weight of HES, a wide range of HES antibody conjugates can be customized.

Antibodies having an increased half-life in vivo can also be generated introducing one or more amino acid modifications (i.e., substitutions, insertions or deletions) into an IgG constant domain, or FcRn binding fragment thereof (preferably a Fc or hinge Fc domain fragment). See, e.g., International Publication No. WO 98/23289; International Publication No. WO 97/34631; and U.S. Pat. No. 6,277,375.

Further, antibodies can be conjugated to albumin in order to make the antibody or antibody fragment more stable in vivo or have a longer half life in vivo. The techniques are well-known in the art, see, e.g., International Publication Nos. WO 93/15199, WO 93/15200, and WO 01/77137; and European Patent No. EP 413,622.

The strategies for increasing half life is especially useful in nanobodies, fibronectin-based binders, and other antibodies or proteins for which increased in vivo half life is desired.

Antibody Conjugates

The present invention provides antibodies or antigen-binding fragments thereof that specifically bind to the extracellular domain of TREM2 (e.g., human TREM2 protein) recombinantly fused or chemically conjugated (including both covalent and non-covalent conjugations) to a heterologous protein or polypeptide (or antigen-binding fragment thereof, preferably to a polypeptide of at least 10, at least 20, at least 30, at least 40, at least 50, at least 60, at least 70, at least 80, at least 90 or at least 100 amino acids) to generate fusion proteins. In particular, the invention provides fusion proteins comprising an antigen-binding fragment of an antibody described herein (e.g., a Fab fragment, Fd fragment, Fv fragment, F (ab)₂ fragment, a VH domain, a VH CDR, a VL domain or a VL CDR) and a heterologous protein, polypeptide, or peptide. Methods for fusing or conjugating proteins, polypeptides, or peptides to an antibody or an antibody fragment are known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International Publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341.

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies and antigen-binding fragments thereof of the invention (e.g., antibodies and antigen-binding fragments thereof with higher affinities and lower dissociation rates). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16 (2):76-82; Hansson, et al., 1999, J. Mol. Biol. 287:265-76; and Lorenzo and Blasco, 1998, Biotechniques 24 (2):308-313 (each of these patents and publications are hereby incorporated by reference in its entirety). Antibodies and antigen-binding fragments thereof, or the encoded antibodies and antigen-binding fragments thereof, may be altered by being subjected to random mutagenesis by error-prone PCR, random nucleotide insertion or other methods prior to recombination. A polynucleotide encoding an antibody antigen-binding fragment thereof that specifically binds to the stalk region of TREM2 may be recombined with one or more components, motifs, sections, parts, domains, fragments, etc. of one or more heterologous molecules.

Moreover, the antibodies and antigen-binding fragments thereof can be fused to marker sequences, such as a peptide to facilitate purification. In one embodiment, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767), and the “FLAG” tag.

In one embodiment, antibodies and antigen-binding fragments thereof of the present invention antigen-binding fragments thereof conjugated to a diagnostic or detectable agent. Such antibodies can be useful for monitoring or prognosing the onset, development, progression and/or severity of a disease or disorder as part of a clinical testing procedure, such as determining the efficacy of a particular therapy. Such diagnosis and detection can accomplished by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (131I, 125I, 123I, and 121I), carbon (14C), sulfur (35S), tritium (3H), indium (115In, 113In, 112In, and 111In), technetium (99Tc), thallium (201Ti), gallium (68Ga, 67Ga), palladium (103Pd), molybdenum (99Mo), xenon (133Xe), fluorine (18F), 153Sm, 177Lu, 159Gd, 149 Pm, 140La, 175Yb, 166Ho, 90Y, 47Sc, 186Re, 188Re, 142Pr, 105Rh, 97Ru, 68Ge, 57Co, 65Zn, 85Sr, 32P, 153Gd, 169Yb, 51Cr, 54Mn, 75Se, 113Sn, and 117Tin; and positron emitting metals using various positron emission tomographies, and nonradioactive paramagnetic metal ions.

Further, an antibody antigen-binding fragment thereof may be conjugated to a therapeutic moiety or drug moiety. Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin; a protein such as tumor necrosis factor, alpha-interferon, beta-interferon, nerve growth factor, platelet derived growth factor, tissue plasminogen activator, an apoptotic agent, an anti-angiogenic agent; or, a biological response modifier such as, for example, a lymphokine.

Moreover, an antibody can be conjugated to therapeutic moieties such as a radioactive metal ion, such as alpha-emitters such as 213Bi or macrocyclic chelators useful for conjugating radiometal ions, including but not limited to, 131In, 131LU, 131Y, 131Ho, 131Sm, to polypeptides. In one embodiment, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4 (10):2483-90; Peterson et al., 1999, Bioconjug. Chem. 10 (4):553-7; and Zimmerman et al., 1999, Nucl. Med. Biol. 26 (8):943-50, each incorporated by reference in their entireties.

Techniques for conjugating therapeutic moieties to antibodies are well known, see, e.g., Amon et al., “Monoclonal Antibodies For Immunotargeting Of Drugs In Cancer Therapy,” in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Nucleic Acids Encoding the Antibodies

The invention provides substantially purified nucleic acid molecules encoding polypeptides comprising segments or domains of the TREM2 antibodies described above. Such polynucleotides can encode at least one CDR region and usually all three CDR regions from the heavy or light chain of the TREM2 antibodies described herein. Such polynucleotides can also encode all or substantially all of the variable region sequence of the heavy chain and/or the light chain of the TREM2 antibodies described herein. Such polynucleotides can also encode both a variable region and a constant region of the antibody. Because of the degeneracy of the code, a variety of nucleic acid sequences will encode each of the immunoglobulin amino acid sequences.

The polynucleotide sequences can be produced by de novo solid-phase DNA synthesis or by PCR mutagenesis of an existing sequence (e.g., sequences as described in the Examples below) encoding an TREM2-binding antibody or its binding fragment. Direct chemical synthesis of nucleic acids can be accomplished by methods known in the art, such as the phosphotriester method of Narang et al., 1979, Meth. Enzymol. 68:90; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, N.Y., 1992; PCR Protocols: A Guide to Methods and Applications, Innis et al. (Ed.), Academic Press, San Diego, Calif., 1990; Mattila et al., Nucleic Acids Res. 19:967, 1991; and Eckert et al., PCR Methods and Applications 1:17, 1991.

Also provided in the invention are expression vectors and host cells for producing the TREM2-binding antibodies described above. Various expression vectors can be employed to express the polynucleotides encoding the TREM2-binding antibody chains or binding fragments. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., Nat Genet. 15:345, 1997). For example, nonviral vectors useful for expression of the TREM2-binding polynucleotides and polypeptides in mammalian (e.g., human) cells include pThioHis A, B & C, pcDNA3.1/His, pEBVHis A, B & C, (Invitrogen, San Diego, Calif.), MPSV vectors, and numerous other vectors known in the art for expressing other proteins. Useful viral vectors include vectors based on retroviruses, adenoviruses, adenoassociated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

The choice of expression vector depends on the intended host cells in which the vector is to be expressed. Typically, the expression vectors contain a promoter and other regulatory sequences (e.g., enhancers) that are operably linked to the polynucleotides encoding an TREM2-binding antibody chain antigen-binding fragment. In one embodiment, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under noninducing conditions without biasing the population for coding sequences whose expression products are better tolerated by the host cells. In addition to promoters, other regulatory elements may also be required or desired for efficient expression of a TREM2-binding antibody chain or antigen-binding fragment. These elements typically include an ATG initiation codon and adjacent ribosome binding site or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

The expression vectors may also provide a secretion signal sequence position to form a fusion protein with polypeptides encoded by inserted TREM2-binding antibody sequences. More often, the inserted TREM2-binding antibody sequences are linked to a signal sequences before inclusion in the vector. Vectors to be used to receive sequences encoding TREM2-binding antibody light and heavy chain variable domains sometimes also encode constant regions or parts thereof. Such vectors allow expression of the variable regions as fusion proteins with the constant regions thereby leading to production of intact antibodies and antigen-binding fragments thereof. Typically, such constant regions are human.

The host cells for harboring and expressing the TREM2-binding antibody chains can be either prokaryotic or eukaryotic. E. coli is one prokaryotic host useful for cloning and expressing the polynucleotides of the present invention. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express TREM2-binding polypeptides of the invention. Insect cells in combination with baculovirus vectors can also be used.

In one embodiment, mammalian host cells are used to express and produce the TREM2-binding polypeptides of the present invention. For example, they can be either a hybridoma cell line expressing endogenous immunoglobulin genes (e.g., the 1D6.C9 myeloma hybridoma clone as described in the Examples) or a mammalian cell line harboring an exogenous expression vector (e.g., the SP2/0 myeloma cells exemplified below). These include any normal mortal or normal or abnormal immortal animal or human cell. For example, a number of suitable host cell lines capable of secreting intact immunoglobulins have been developed including the CHO cell lines, various Cos cell lines, HeLa cells, myeloma cell lines, transformed B-cells and hybridomas. The use of mammalian tissue cell culture to express polypeptides is discussed generally in, e.g., Winnacker, FROM GENES TO CLONES, VCH Publishers, N.Y., N.Y., 1987. Expression vectors for mammalian host cells can include expression control sequences, such as an origin of replication, a promoter, and an enhancer (see, e.g., Queen, et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP poIIII promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts. (See generally Sambrook, et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express TREM2-binding antibody chains or binding fragments can be prepared using expression vectors of the invention which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type.

Generation of Monoclonal Antibodies

Monoclonal antibodies (mAbs) can be produced by a variety of techniques, including conventional monoclonal antibody methodology, e.g., the standard somatic cell hybridization technique of Kohler and Milstein, 1975 Nature 256: 495. Many techniques for producing monoclonal antibody can be employed e.g., viral or oncogenic transformation of B lymphocytes.

An animal system for preparing hybridomas is the murine system. Hybridoma production in the mouse is a well established procedure Immunization protocols and techniques for isolation of immunized splenocytes for fusion are known in the art. Fusion partners (e.g., murine myeloma cells) and fusion procedures are also known.

In some embodiments, the antibodies of the invention are humanized monoclonal antibodies. Chimeric or humanized antibodies and antigen-binding fragments thereof of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art. See e.g., U.S. Pat. No. 5,225,539 to Winter, and U.S. Pat. Nos. 5,530,101; 5,585,089; 5,693,762 and 6,180,370 to Queen et al.

In some embodiments, the antibodies of the invention are human monoclonal antibodies. Such human monoclonal antibodies directed against TREM2 can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.”

The HuMAb Mouse® (Medarex, Inc.) contains human immunoglobulin gene miniloci that encode un-rearranged human heavy (mu and gamma) and kappa light chain immunoglobulin sequences, together with targeted mutations that inactivate the endogenous mu and kappa chain loci (see e.g., Lonberg, et al., 1994 Nature 368 (6474): 856-859). Accordingly, the mice exhibit reduced expression of mouse IgM or K, and in response to immunization, the introduced human heavy and light chain transgenes undergo class switching and somatic mutation to generate high affinity human IgG-kappa monoclonal (Lonberg, N. et al., 1994 supra; reviewed in Lonberg, N., 1994 Handbook of Experimental Pharmacology 113:49-101; Lonberg, N. and Huszar, D., 1995 Intern. Rev. Immunol. 13: 65-93, and Harding, F. and Lonberg, N., 1995 Ann. N.Y. Acad. Sci. 764:536-546). The preparation and use of HuMAb mice, and the genomic modifications carried by such mice, is further described in Taylor, L. et al., 1992 Nucleic Acids Research 20:6287-6295; Chen, J. et al., 1993 International Immunology 5: 647-656; Tuaillon et al., 1993 Proc. Natl. Acad. Sci. USA 94:3720-3724; Choi et al., 1993 Nature Genetics 4:117-123; Chen, J. et al., 1993 EMBO J. 12: 821-830; Tuaillon et al., 1994 J. Immunol. 152:2912-2920; Taylor, L. et al., 1994 International Immunology 579-591; and Fishwild, D. et al., 1996 Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92103918, WO 93/12227, WO 94/25585, WO 97113852, WO 98/24884 and WO 99/45962, all to Lonberg and Kay; and PCT Publication No. WO 01/14424 to Korman et al.

In some embodiments, human antibodies can be raised using a mouse that carries human immunoglobulin sequences on transgenes and transchomosomes such as a mouse that carries a human heavy chain transgene and a human light chain transchromosome. Such mice, referred to herein as “KM mice,” are described in detail in PCT Publication WO 02/43478 to Ishida et al.

Still further, alternative transgenic animal systems expressing human immunoglobulin genes are available in the art and can be used to raise TREM2-binding antibodies and antigen-binding fragments thereof. For example, an alternative transgenic system referred to as the Xenomouse (Abgenix, Inc.) can be used. Such mice are described in, e.g., U.S. Pat. Nos. 5,939,598; 6,075,181; 6,114,598; 6,150,584 and 6,162,963 to Kucherlapati et al.

Moreover, alternative transchromosomic animal systems expressing human immunoglobulin genes are available in the art and can be used to raise TREM2-binding antibodies of the invention. For example, mice carrying both a human heavy chain transchromosome and a human light chain transchromosome, referred to as “TC mice” can be used; such mice are described in Tomizuka et al., 2000 Proc. Natl. Acad. Sci. USA 97:722-727. Furthermore, cows carrying human heavy and light chain transchromosomes have been described in the art (Kuroiwa et al., 2002 Nature Biotechnology 20:889-894) and can be used to raise TREM2-binding antibodies of the invention.

Human monoclonal antibodies can also be prepared using phage display methods for screening libraries of human immunoglobulin genes. Such phage display methods for isolating human antibodies are established in the art or described in the examples below. See for example: U.S. Pat. Nos. 5,223,409; 5,403,484; and U.S. Pat. No. 5,571,698 to Ladner et al; U.S. Pat. Nos. 5,427,908 and 5,580,717 to Dower et al; U.S. Pat. Nos. 5,969,108 and 6,172,197 to McCafferty et al; and U.S. Pat. Nos. 5,885,793; 6,521,404; 6,544,731; 6,555,313; 6,582,915 and 6,593,081 to Griffiths et al.

Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

Framework or Fc Engineering

Engineered antibodies and antigen-binding fragments thereof of the invention include those in which modifications have been made to framework residues within VH and/or VL, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis. Such “backmutated” antibodies are also intended to be encompassed by the invention.

Another type of framework modification involves mutating one or more residues within the framework region, or even within one or more CDR regions, to remove T cell-epitopes to thereby reduce the potential immunogenicity of the antibody. This approach is also referred to as “deimmunization” and is described in further detail in U.S. Patent Publication No. 20030153043 by Carr et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half-life. Various approaches are possible. For example, one or more of the following mutations can be introduced: T252L, T254S, T256F, as described in U.S. Pat. No. 6,277,375 to Ward. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In one embodiment, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another embodiment, one or more amino acids selected from amino acid residues can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another embodiment, one or more amino acid residues are altered to thereby alter the ability of the antibody to fix complement. This approach is described further in PCT Publication WO 94/29351 by Bodmer et al.

In some embodiments, the TREM2-binding molecule contains a human IgG1 constant region. In some embodiments, the human IgG1 constant region includes an Fc region.

In some embodiments, the Fc region of the TREM2-binding molecule includes one or more mutations mediating reduced or no antibody-dependent cellular cytotoxicity (ADCC) or complement-dependent cytotoxicity (CDC). In some embodiments, amino acid residues L234 and L235 of the IgG1 constant region are substituted to A234 and A235. In some embodiments, amino acid residue N267 of the IgG1 constant region is substituted to A267. In some embodiments, amino acid residues D265 and P329 of the IgG1 constant region are substituted to A265 and A329. In certain embodiments, the Fc region optionally comprises a mutation or combination of mutations conferring reduced effector function selected from any of D265A, P329A, P329G, N297A, D265A/P329A, D265A/N297A, L234/L235A, P329A/L234A/L235A, and P329G/L234A/L235A. In some embodiments, the Fc region comprises a mutation or combination of mutations conferring reduced effector function selected from any of D265A, P329A, P329G, N297A, D265A/P329A, D265A/N297A, L234/L235A, P329A/L234A/L235A, and P329G/L234A/L235A (all positions by EU numbering).

In yet another embodiment, the Fc region is modified to increase the ability of the antibody to mediate antibody dependent cellular cytotoxicity (ADCC) and/or to increase the affinity of the antibody for an Fc-gamma receptor by modifying one or more amino acids. This approach is described further in PCT Publication WO 00/42072 by Presta. Moreover, the binding sites on human IgG1 for Fc-gamma RI, Fc-gamma RII, Fc-gamma RIII and FcRn have been mapped and variants with improved binding have been described (see Shields, R. L. et al., 2001 J. Biol. Chen. 276:6591-6604). For example, the Fc region can comprise a mutation or combination of mutations conferring increased effector function selected from any of S239D, 1332E, A330L, S298A, E333A, E333S, K334A, K236A, K236W, F243L, P247I, D280H, K290S, R292P, S298D, S298V, Y300L, V305I, A339D, A339Q, A339T, P396L (all positions by EU numbering).

In still another embodiment, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, LecI3 cells, with reduced ability to attach fucose to Asn (297)-linked carbohydrates, also resulting in hypofucosylation of antibodies expressed in that host cell (see also Shields, R. L. et al., 2002 J. Biol. Chem. 277:26733-26740). PCT Publication WO 99/54342 by Umana et al. describes cell lines engineered to express glycoprotein-modifying glycosyl transferases (e.g., beta (1,4)-N acetylglucosaminyltransferase III (GnTIII)) such that antibodies expressed in the engineered cell lines exhibit increased bisecting GlcNac structures which results in increased ADCC activity of the antibodies (see also Umana et al., 1999 Nat. Biotech. 17:176-180).

In some embodiments, the TREM2-binding molecule is an antibody. In some embodiments, the antibody has an IgG1 isotype with one or more mutations (e.g., relative to a wild-type Fc region of the same isotype). In some embodiments, the one or more mutations are selected from N297A, N297Q (BoltS et al. (1993) Eur J Immunol 23:403-411), D265A, L234A, L235A (McEarchern et al., (2007) Blood, 109:1185-1192), C226S, C229S (McEarchern et al., (2007) Blood, 109:1185-1192), P238S (Davis et al., (2007) J Rheumatol, 34:2204-2210), E233P, L234V (McEarchern et al., (2007) Blood, 109:1185-1192), P238A, A327Q, A327G, P329A (Shields RL. et al., (2001) J Bioi Chern. 276(9):6591-604), K322A, L234F, L235E (Hezareh, et al., (2001) J Viral 75, 12161-12168; Oganesyan et al., (2008). Acta Crystallographica 64, 700-704), P331S (Oganesyan et al., (2008) Acta Crystallographica 64, 700-704), T394D (Wilkinson et al. (2013) MAbs 5(3): 406-417), A330L, M252Y, S254T, and/or T256E, where the amino acid position is according to the EU or Kabat numbering convention. In certain embodiments, the Fc region further includes an amino acid deletion at a position corresponding to glycine 236 according to the EU or Kabat numbering convention.

In some embodiments, the antibody has an IgG1 isotype with a heavy chain constant region that contains a C220S mutation according to the EU or Kabat numbering convention.

In some embodiments, the Fc region further contains one or more additional mutations selected from A330L, L234F; L235E, and/or P331S according to EU or Kabat numbering convention.

In certain embodiments, the antibody has an IgG2 isotype. In some embodiments, the antibody contains a human IgG2 constant region. In some embodiments, the human IgG2 constant region includes an Fc region. In some embodiments, the Fc region contains one or more modifications. For example, in some embodiments, the Fc region contains one or more mutations (e.g., relative to a wild-type Fc region of the same isotype). In some embodiments, the one or more mutations are selected from V234A, G237A, H268E, V309L, N297A, N297Q, A330S, P331S, C232S, C233S, M252Y, S254T, and/or T256E, where the amino acid position is according to the EU or Kabat numbering convention.

In certain embodiments, the antibody has an IgG4 isotype. In some embodiments, the antibody contains a human IgG4 constant region. In some embodiments, the human IgG4 constant region includes an Fc region. In some embodiments, the Fc region contains one or more modifications. For example, in some embodiments, the Fc region contains one or more mutations (e.g., relative to a wild-type Fc region of the same isotype). In some embodiments, the one or more mutations are selected from E233P, F234V, L235A, G237A, E318A (Hutchins et al. (1995) Proc Nat/A cad Sci USA, 92:11980-11984), S228P, L236E, S241P, L248E (Reddy et al., (2000) J Immuno/, 164:1925-1933; Angal et al., (1993) Mol Immunol. 30(1):105-8; U.S. Pat. No. 8,614,299 B2), T394D, M252Y, S254T, T256E, N297A, and/or N297Q, where the amino acid position is according to the EU or Kabat numbering convention.

In some embodiments, the Fc region further contains one or more additional mutations selected from a M252Y, S254T, and/or T256E, where the amino acid position is according to the EU or Kabat numbering convention.

In some embodiments, one or more of the IgG1 variants described herein may be combined with an A330L mutation (Lazar et al., (2006) Proc Natl Acad Sci USA, 103:4005-4010), or one or more of L234F, L235E, and/or P331S mutations (Sazinsky et al., (2008) Proc Natl Acad Sci USA, 105:20167-20172), where the amino acid position is according to the EU or Kabat numbering convention, to eliminate complement activation. In some embodiments, the IgG variants described herein may be combined with one or more mutations to enhance the antibody half-liFc in human serum (e.g. M252Y, S254T, T256E mutations according to the EU or Kabat numbering convention) (Dall'Acqua et al., (2006) J Biol Chern, 281:23514-23524; and Strohl e al., (2009) Current Opinion in Biotechnology, 20:685-691).

In some embodiments, an IgG4 variant of the present disclosure may be combined with an S228P mutation according to the EU or Kabat numbering convention (Angal et al., (1993) Mol Immunol, 30:105-108) and/or with one or more mutations described in Peters et al., (2012) J Biol Chern. 13; 287(29):24525-33) to enhance antibody stabilization.

In some embodiments, the antibody has an Fc region selected from an IgG2 Fc region, an IgG4 Fc region, or an IgG2/IgG4 hybrid Fc region.

Methods of Engineering Altered Antibodies

As discussed above, the TREM2-binding antibodies having VH and VL sequences or full length heavy and light chain sequences shown herein can be used to create new TREM2-binding antibodies by modifying full length heavy chain and/or light chain sequences, VH and/or VL sequences, or the constant region (s) attached thereto. Thus, in another aspect of the invention, the structural features of TREM2-binding antibody of the invention are used to create structurally related TREM2-binding antibodies that retain at least one functional property of the antibodies and antigen-binding fragments thereof of the invention, such as binding to and stabilize human TREM2.

For example, one or more CDR regions of the antibodies and antigen-binding fragments thereof of the present invention, or mutations thereof, can be combined recombinantly with known framework regions and/or other CDRs to create additional, recombinantly-engineered, TREM2-binding antibodies and antigen-binding fragments thereof of the invention, as discussed above. Other types of modifications include those described in the previous section. The starting material for the engineering method is one or more of the VH and/or VL sequences provided herein, or one or more CDR regions thereof. To create the engineered antibody, it is not necessary to actually prepare (i.e., express as a protein) an antibody having one or more of the VH and/or VL sequences provided herein, or one or more CDR regions thereof. Rather, the information contained in the sequence (s) is used as the starting material to create a “second generation” sequence (s) derived from the original sequence (s) and then the “second generation” sequence (s) is prepared and expressed as a protein.

The altered antibody sequence can also be prepared by screening antibody libraries having fixed CDR3 sequences or minimal essential binding determinants as described in US20050255552 and diversity on CDR1 and CDR2 sequences. The screening can be performed according to any screening technology appropriate for screening antibodies from antibody libraries, such as phage display technology.

Standard molecular biology techniques can be used to prepare and express the altered antibody sequence. The antibody encoded by the altered antibody sequence (s) is one that retains one, some or all of the functional properties of the TREM2-binding antibodies described herein, which functional properties include, but are not limited to, specifically binding to and stabilize human TREM2 protein.

The functional properties of the altered antibodies can be assessed using standard assays available in the art and/or described herein, such as those set forth in the Examples (e.g., ELISAs).

In some embodiments, the methods of engineering antibodies and antigen-binding fragments thereof of the invention, mutations can be introduced randomly or selectively along all or part of an TREM2-binding antibody coding sequence and the resulting modified TREM2-binding antibodies can be screened for binding activity and/or other functional properties as described herein. Mutational methods have been described in the art. For example, PCT Publication WO 02/092780 by Short describes methods for creating and screening antibody mutations using saturation mutagenesis, synthetic ligation assembly, or a combination thereof. Alternatively, PCT Publication WO 03/074679 by Lazar et al. describes methods of using computational screening methods to optimize physiochemical properties of antibodies.

Characterization of the Antibodies of the Invention

The antibodies and antigen-binding fragments thereof of the invention can be characterized by various functional assays. For example, they can be characterized by their ability to bind and stabilize TREM2 protein (e.g., human TREM2 protein).

The ability of an antibody to bind to TREM2 (e.g., human TREM2 protein) can be detected by labelling the antibody of interest directly, or the antibody may be unlabeled and binding detected indirectly using various sandwich assay formats known in the art.

In some embodiments, the TREM2-binding antibodies and antigen-binding fragments thereof of the invention block or compete with binding of a reference TREM2-binding antibody to TREM2 protein (e.g., human TREM2 protein). These can be fully human or humanized TREM2-binding antibodies described above. They can also be other human, mouse, chimeric or humanized TREM2-binding antibodies which bind to the same epitope as the reference antibody. The capacity to block or compete with the reference antibody binding indicates that TREM2-binding antibody under test binds to the same or similar epitope as that defined by the reference antibody, or to an epitope which is sufficiently proximal to the epitope bound by the reference TREM2-binding antibody. Such antibodies are especially likely to share the advantageous properties identified for the reference antibody. The capacity to block or compete with the reference antibody may be determined by, e.g., a competition binding assay. With a competition binding assay, the antibody under test is examined for ability to inhibit specific binding of the reference antibody to a common antigen, such as TREM2 protein (e.g., human TREM2 protein). A test antibody competes with the reference antibody for specific binding to the antigen if an excess of the test antibody substantially inhibits binding of the reference antibody. Substantial inhibition means that the test antibody reduces specific binding of the reference antibody usually by at least 10%, 25%, 50%, 75%, or 90%.

There are a number of known competition binding assays that can be used to assess competition of an antibody with a reference antibody for binding to a particular protein, in this case, TREM2 (e.g., human TREM2 protein). These include, e.g., solid phase direct or indirect radioimmunoassay (RIA), solid phase direct or indirect enzyme immunoassay (EIA), sandwich competition assay (see Stahli et al., Methods in Enzymology 9:242-253, 1983); solid phase direct biotin-avidin EIA (see Kirkland et al., J. Immunol. 137:3614-3619, 1986); solid phase direct labeled assay, solid phase direct labeled sandwich assay (see Harlow & Lane, supra); solid phase direct label RIA using 1-125 label (see Morel et al., Molec. Immunol. 25:7-15, 1988); solid phase direct biotin-avidin EIA (Cheung et al., Virology 176:546-552, 1990); and direct labeled RIA (Moldenhauer et al., Scand. J. Immunol. 32:77-82, 1990). Typically, such an assay involves the use of purified antigen bound to a solid surface or cells bearing either of these, an unlabelled test TREM2-binding antibody and a labelled reference antibody. Competitive inhibition is measured by determining the amount of label bound to the solid surface or cells in the presence of the test antibody. Usually the test antibody is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

To determine if the selected TREM2-binding monoclonal antibodies bind to unique epitopes, each antibody can be biotinylated using commercially available reagents (e.g., reagents from Pierce, Rockford, Ill.). Competition studies using unlabeled monoclonal antibodies and biotinylated monoclonal antibodies can be performed using TREM2 protein coated-ELISA plates. Biotinylated MAb binding can be detected with a strep-avidin-alkaline phosphatase probe. To determine the isotype of a purified TREM2-binding antibody, isotype ELISAs can be performed. For example, wells of microtiter plates can be coated with 1 μg/ml of anti-human IgG overnight at 4 degrees C. After blocking with 1% BSA, the plates are reacted with 1 μg/ml or less of the monoclonal TREM2-binding antibody or purified isotype controls, at ambient temperature for one to two hours. The wells can then be reacted with either human IgG1 or human IgM-specific alkaline phosphatase-conjugated probes. Plates are then developed and analyzed so that the isotype of the purified antibody can be determined.

To demonstrate binding of monoclonal TREM2-binding antibodies to live cells expressing TREM2 protein (e.g., human TREM2 protein), flow cytometry can be used. Briefly, cell lines expressing TREM2 (grown under standard growth conditions) can be mixed with various concentrations of TREM2-binding antibody in PBS containing 0.1% BSA and 10% fetal calf serum, and incubated at 37 degrees ° C. for 1 hour. After washing, the cells are reacted with Fluorescein-labeled anti-human IgG antibody under the same conditions as the primary antibody staining. The samples can be analyzed by FACScan instrument using light and side scatter properties to gate on single cells. An alternative assay using fluorescence microscopy may be used (in addition to or instead of) the flow cytometry assay. Cells can be stained exactly as described above and examined by fluorescence microscopy. This method allows visualization of individual cells, but may have diminished sensitivity depending on the density of the antigen.

TREM2-binding antibodies and antigen-binding fragments thereof of the invention can be further tested for reactivity with TREM2 protein (e.g., human TREM2 protein) or antigenic fragment by Western blotting. Briefly, purified TREM2 protein (e.g., human TREM2 protein) or fusion proteins, or cell extracts from cells expressing TREM2 can be prepared and subjected to sodium dodecyl sulfate polyacrylamide gel electrophoresis. After electrophoresis, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

Examples of functional assays are also described in the Example section below.

Aptamers

Provided herein are aptamers that specifically bind to the extracellular domain of TREM2 protein (e.g., human TREM2 protein) and stabilize the TREM2 protein (e.g., human TREM2 protein). In some embodiments, those aptamers specifically bind to the extracellular domain of human TREM2, e.g., the amino acid residues 14 to 174 of SEQ ID NO: 1, the amino acid residues 14 to 168 of SEQ ID NO: 3, or the amino acid residues 14 to 171 of SEQ ID NO: 4. In some embodiments, those aptamers specifically bind to a stalk region of human TREM2, e.g., having an amino acid sequence of any of SEQ ID NO: 7, 8, or 9. Those aptamers can stabilize the human TREM2 protein on the cell surface, and/or reduce shedding of the ectodomain of the human TREM2 protein.

Aptamers are usually created by selection of a large random sequence pool, but natural aptamers also exist. Modulation of the target molecule by an aptamer may occur by binding to the target, by catalytically altering the target, by reacting with the target in a way that modifies/alters the target or the functional activity of the target, by covalently attaching to the target as a suicide inhibitor, by facilitating the reaction between the target and another molecule.

Oligonucleotide aptamers may be comprised of multiple ribonucleotide units, deoxyribonucleotide units, or a mixture of those units. Oligonucleotide aptamers may further comprise one or more modified bases, sugars, phosphate backbone units. Peptide aptamers are small, highly stable proteins that provide a high affinity binding surface for a specific target protein. They usually consist of a protein scaffold with variable peptide loops attached at both ends. The variable loop is typically composed of ten to twenty amino acids, and the scaffold can be any protein that has good solubility and compacity properties. This double structural constraint greatly increases the binding affinity of the peptide aptamer to its target protein.

In vitro selection of aptamers that bind with high specificity to a target molecule such as the stalk region of human TREM2 protein, can be performed using the SELEX process as described in U.S. Pat. Nos. 5,475,096; 5,270,163; 5,580,737; 5,567,588; 5,496,938; 5,705,337; 6,376,424; 5,707,796; all of which are incorporated by reference herein. The SELEX process encompasses the identification of high-affinity aptamers containing modified nucleotides conferring improved characteristics on the ligand, such as improved in vivo stability or improved delivery characteristics. Examples of such modifications include chemical substitutions at the ribose and/or phosphate and/or base positions. SELEX process-identified aptamers containing modified nucleotides are described in U.S. Pat. Nos. 5,660,985 and 5,580,737, each of which is incorporated by reference herein. Further modifications of the SELEX process are described in U.S. Pat. Nos. 5,763,177; 6,001,577; 6,291,184; 6,458,539; all of which are incorporated by reference herein. Aptamers that specifically bind to a stalk region of human TREM2 protein, e.g., having an amino acid sequence of any of SEQ ID NO: 7, 8, or 9, can be generated using any of the SELEX process described above.

Slow off-rate modified aptamers (SOMAmers) can be generated using improved SELEX methods that include a slow off-rate enrichment process as described in U.S. Pat. Nos. 7,964,356 and 8,975,026, each of which is incorporated by reference herein. During the slow off-rate enrichment process, the relative concentration of aptamer affinity complexes having slow dissociation rates is increased relative to the concentration of aptamer affinity complexes having faster, less desirable dissociation rates. The slow off-rate enrichment process can be a solution-based slow off-rate enrichment process that takes place in solution. The slow off-rate enrichment process can include one or more of the following steps: (1) the addition of a competitor molecule, e.g., a polyanion (e.g., heparin or dextran sulfate (dextran)), after the target/aptamer complexes have been allowed to form, (2) dilution of the mixture, or (3) a combination of (1) and (2), e.g., dilution of the mixture in the presence of a competitor molecule. Because the effect of an slow off-rate enrichment process generally depends on the differing dissociation rates of different aptamer affinity complexes (i.e., aptamer affinity complexes formed between the target molecule and different nucleic acids in the candidate mixture), the duration of the slow off-rate enrichment process is selected so as to retain a high proportion of aptamer affinity complexes having slow dissociation rates while substantially reducing the number of aptamer affinity complexes having fast dissociation rates. The slow off-rate enrichment process may be used in one or more cycles during the SELEX process. When dilution and the addition of a competitor are used in combination, they may be performed simultaneously or sequentially, in any order. The slow off-rate enrichment process can be used when the total concentration of the target protein in the mixture is low. In one embodiment, when the slow off-rate enrichment process includes dilution, the mixture can be diluted as much as is practical. SOMAmers that specifically bind to the stalk region of human TREM2 protein, e.g., having an amino acide sequence of any of SEQ ID NO: 7, 8, or 9, can be generated using any of the improved SELEX methods that include a slow off-rate enrichment process as described above.

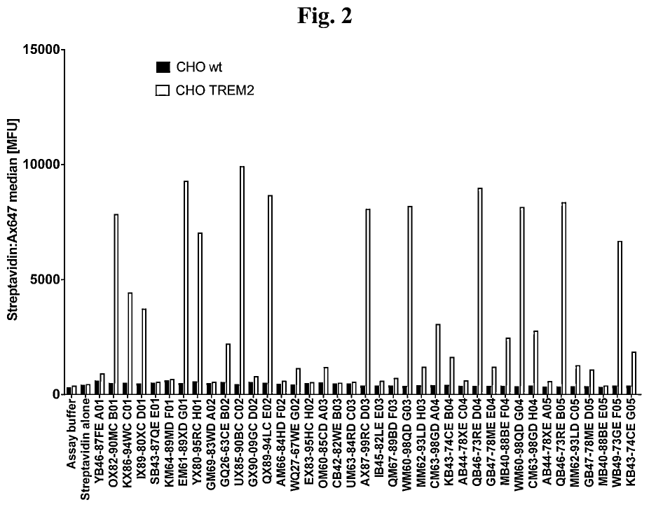

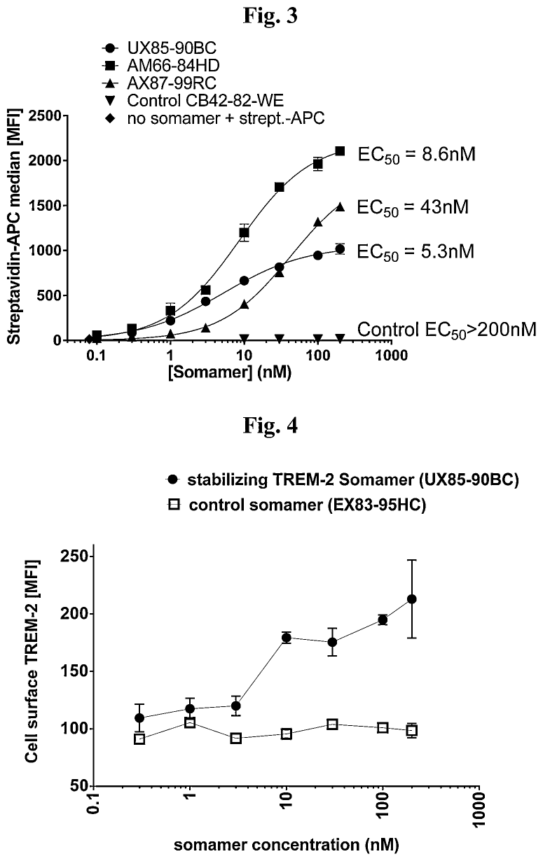

The data presented herein showed that SOMAmers that direct bind to the extracellular domain of human TREM2 can stabilize cell surface TREM2 level and increase the cell surface expression of TREM2 over time (see FIGS. 4, 5A and 5B).

Low Molecular Weight Compounds

Also provided are low molecular weight compounds, e.g., compounds having a molecular weight of less than or equal to 2000 Da, that specifically bind to the extracellular domain of TREM2 protein (e.g., human TREM2 protein) and stabilize the TREM2 protein (e.g., human TREM2 protein). In some embodiments, those low molecular weight compounds specifically bind to the extracellular domain of human TREM2, e.g., the amino acid residues 14 to 174 of SEQ ID NO: 1, the amino acid residues 14 to 168 of SEQ ID NO: 3, or the amino acid residues 14 to 171 of SEQ ID NO: 4. In some embodiments, those low molecular weight compounds specifically bind to a stalk region of human TREM2, e.g., having an amino acid sequence of any of SEQ ID NO: 7, 8, or 9. Those low molecular weight compounds can stabilize the human TREM2 protein on the cell surface, and/or reduce shedding of the ectodomain of the human TREM2 protein.

Methods of Treatment

Provided herein are methods of treating a disease associated with TREM2 loss of function (e.g., human TREM2 loss of function) by using the TREM2-binding molecules (e.g., human TREM2-binding molecules) disclosed herein. In some embodiments, the disease associated with TREM2 loss of function (e.g., human TREM2 loss of function) is a neuroinflammatory or neurodegenerative disease such as Alzheimer's disease, frontotemporal dementia, Parkinson's disease, amyotrophic lateral sclerosis, Nasu-Hakola disease, multiple sclerosis, amyotrophic lateral sclerosis (ALS), anti-NMDA receptor encephalitis, autism, brain lupus (NP-SLE), chemo-induced peripheral neuropathy (CIPN), postherapeutic neuralgia, chronic inflammatory demyelinating polyneuropathy (CIDP), epilepsy, Guillain-Barré Syndrom (GBS), inclusion body myositis, lysosomal storage diseases, e.g., sphingomyelinlipidose (Niemann-Pick C) and mucopolysaccharidose II/IIIB, metachromatic leukodystrophy, multifocal motor neuropathy, Myasthenia Gravis, Neuro-Behcet's Disease, neuromyelitis optica (NMO), optic neuritis, polymyositis, dermatomyositis, Rasmussen's encephalitis, Rett's Syndrome, stroke, transverse myelitis, traumatic brain injury, spinal cord injury, viral encephalitis, or bacterial meningitis.

Provided herein are also methods of treating a TREM2 related disorder (e.g., a human TREM2 related disorder) directly or indirectly associated with aberrant TREM2 activity and/or expression by using the TREM2-binding molecules (e.g., human TREM2-binding molecules) disclosed herein. The Trem2-related disorders (e.g., a human TREM2 related disorder) include CNS related diseases, PNS related diseases, systemic inflammation and other diseases related to inflammation, pain and withdrawal symptoms caused by an abuse of chemical substances, diseases or disorders related to the CNS include general anxiety disorders, cognitive disorders, learning and memory deficits and dysfunctions, Alzheimer's disease (mild, moderate and severe), attention deficit and hyperactivity disorder, Parkinson's disease, dementia in Parkinson's disease, Huntington's disease, ALS, prionic neurodegenerative disorders such as Creutzfeld-Jacob disease and kuru disease, Gilles de la Tourette's syndrome, psychosis, depression and depressive disorders, mania, manic depression, schizophrenia, the cognitive deficits in schizophrenia, obsessive compulsive disorders, panic disorders, eating disorders, narcolepsy, nociception, AIDS-dementia, senile dementia, mild cognitive impairment related to age (MCI), age associated memory impairment, autism, dyslexia, tardive dyskinesia, epilepsy, and convulsive disorders, post-traumatic stress disorders, transient anoxia, pseudodementia, pre-menstrual syndrome, late luteal phase syndrome, chronic fatigue syndrome and jet lag.

Trem2-related disorders (e.g., human TREM2 related disorders) also include: immunological disorders, especially involving inflammatory disorders (e.g., bacterial infection, fungal infection, viral infection, protozoa or other parasitic infection, psoriasis, septicemia, cerebral malaria, inflammatory bowel disease, arthritis, such as rheumatoid arthritis, folliculitis, impetigo, granulomas, lipoid pneumonias, vasculitis, and osteoarthritis), autoimmune disorders (e.g., rheumatoid arthritis, thyroiditis, such as Hashimoto's thyroiditis and Graves' disease, insulin-resistant diabetes, pernicious anemia, Addison's disease, pemphigus, vitiligo, ulcerative colitis, systemic lupus erythematosus (SLE), Sjogren's syndrome, multiple sclerosis, dermatomyositis, mixed connective tissue disease, scleroderma, polymyositis, graft rejection, such as allograft rejection), T cell disorders (e.g., AIDS), allergic inflammatory disorders (e.g., skin and/or mucosal allergies, such as allergic rhinitis, asthma, psoriasis), neurological disorders, eye disorders, embryonic disorders, or any other disorders (e.g., tumors, cancers, leukemia, myeloid diseases, and traumas) which are directly or indirectly associated with aberrant TREM2 activity and/or expression.

In some embodiments, the Trem2-related disorder (e.g., human TREM2 related disorder) is an autoimmune, inflammatory, or malignant disorder mediated by or associated with extensive proteolytic cleavage of TREM2 or cells expressing aberrant or mutated variants of the TREM2 receptor. Examples of autoimmune diseases include, without limitation, arthritis (for example rheumatoid arthritis, arthritis chronica progrediente and arthritis deformans) and rheumatic diseases, including inflammatory conditions and rheumatic diseases involving bone loss, inflammatory pain, spondyloarhropathies including ankolsing spondylitis, Reiter syndrome, reactive arthritis, psoriatic arthritis, and enterophathis arthritis, hypersensitivity (including both airways hypersensitivity and dermal hypersensitivity) and allergies. Autoimmune diseases include autoimmune haematological disorders (including e.g. hemolytic anaemia, aplastic anaemia, pure red cell anaemia and idiopathic thrombocytopenia), systemic lupus erythematosus, inflammatory muscle disorders, polychondritis, sclerodoma, Wegener granulomatosis, dermatomyositis, chronic active hepatitis, myasthenia gravis, psoriasis, Steven-Johnson syndrome, idiopathic sprue, endocrine ophthalmopathy, Graves disease, sarcoidosis, multiple sclerosis, primary biliary cirrhosis, juvenile diabetes (diabetes mellitus type I), uveitis (anterior and posterior), keratoconjunctivitis sicca andvemal keratoconjunctivitis, interstitial lung fibrosis, psoriatic arthritis and glomerulonephritis (with and without nephrotic syndrome, e.g. including gout, langerhans cell histiocytosis, idiopathic nephrotic syndrome or minimal change nephropathy), tumors, inflammatory disease of skin and cornea, myositis, loosening of bone implants, metabolic disorders, such as atherosclerosis, diabetes, and dislipidemia.

In some embodiments, the Trem2-related disorder (e.g., human TREM2 related disorder) is selected from asthma, bronchitis, pneumoconiosis, pulmonary emphysema, other obstructive or inflammatory diseases of the airways including idiopathic pulmonary fibrosis or COPD.

In some embodiments, the Trem2-related disorder (e.g., human TREM2 related disorder) is a hematopoietic or hepatopoetic malignant disorder such as acute myeloid leukemia, chronic myeloid leukemia, myeloproliferative disorders, myelodysplastic syndromes, multiple myeloma, paroxysmal nocturnal hemoglobinuria, fanconi anemi, thalassemia major, Wiskott-Aldrich syndrome, hemophagocytic lymphohistiocytosis.

In some embodiments, the Trem2-related disorder (e.g., human TREM2 related disorder) is selected from asthma, encephalitis, inflammatory bowel disease, chronic obstructive pulmonary disease (COPD), allergic disorders, septic shock, pulmonary fibrosis, undifferentiated spondyloarthropathy, undifferentiated arthropathy, arthritis, inflammatory osteolysis, or chronic inflammation resulting from chronic viral or bacterial infections.

In some embodiments, the Trem2-related disorder (e.g., human TREM2 related disorder) is selected from dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, Taupathy disease, Nasu-Hakola disease, stroke, acute trauma, chronic trauma, lupus, acute and chronic colitis, wound healing, Crohn's disease, inflammatory bowel disease, ulcerative colitis, obesity, Malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, Sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, and cancer.

In some embodiments, the Trem2-related disorder (e.g., human TREM2 related disorder) is selected from dementia, frontotemporal dementia, Alzheimer's disease, Nasu-Hakola disease, and multiple sclerosis. In some embodiments, Trem2-related disorder is a dementia such as frontotemporal dementia, Alzheimer's disease, vascular dementia, semantic dementia, or dementia with Lewy bodies.

In some embodiments, such methods include administering to a subject in need of treatment a therapeutically effective amount of a molecule that specifically binds to the extracellular domain of TREM2 protein (e.g., human TREM2 protein) and stabilizes the TREM2 protein (e.g., human TREM2 protein). In some embodiments, the TREM2-binding molecule specifically binds to the extracellular domain of human TREM2, e.g., the amino acid residues 14 to 174 of SEQ ID NO: 1, the amino acid residues 14 to 168 of SEQ ID NO: 3, or the amino acid residues 14 to 171 of SEQ ID NO: 4. In some embodiments, the TREM2-binding molecule specifically binds to a stalk region of human TREM2, e.g., having an amino acid sequence of any of SEQ ID NO: 7, 8, or 9.

In some embodiments, such methods of treating a disease associated with TREM2 loss of function (e.g., human TREM2 loss of function) include: (1) assaying the cell surface TREM2 level (e.g., human TREM2 level) in a sample obtained from a subject, e.g., a cerebrospinal fluid sample obtained from a subject; (2) selecting a subject whose level of cell surface TREM2 (e.g., human TREM2 level) is lower than a reference level, wherein the reference level is the level of cell surface TREM2 (e.g., human TREM2 level) in a sample obtained from a healthy subject, e.g., e.g., a cerebrospinal fluid sample obtained from a healthy subject; and (3) administering to the selected subject a therapeutically effective amount of a molecule that specifically binds to the extracellular domain of TREM2 protein (e.g., human TREM2 protein) and stabilizes the TREM2 protein (e.g., human TREM2 protein). The molecules that specifically bind to the extracellular domain of TREM2 protein (e.g., human TREM2 protein) can stabilize the TREM2 protein (e.g., human TREM2 protein) on the cell surface, and/or reduce shedding of the ectodomain of the TREM2 protein (e.g., human TREM2 protein). These molecules can be administered to the subject through an oral, intravenous, intracranial, intrathecal, subcutaneous or intranasal route. The level of cell surface TREM2 (e.g., human TREM2) in the sample can be determined by an assay known in the art, e.g., by flow cytometry, immunohistochemistry, Western blotting, immunofluorescent assay, radioimmunoassay (RIA), enzyme-linked immunosorbent assay (ELISA), homogeneous time resolved fluorescence (HTRF), positron emission tomography (PET), or any other immune detection with an antibody or antibody fragment against TREM2 protein (e.g., human TREM2 protein).

Combination Therapies

The various treatments described above can be combined with other treatment partners such as the current standard of care for a disease associated with TREM2 loss of function (e.g., human TREM2 loss of function), e.g., the current standard of care for Alzheimer's disease, frontotemporal dementia, Parkinson's disease, amyotrophic lateral sclerosis, or Nasu-Hakola disease. For example, the TREM2-binding molecules (e.g., human TREM2-binding molecules) described herein can be combined with one or more of BACE inhibitors, anti-Tau antibodies, anti-amyloid beta antibodies, FTY720, B G12, interferon beta or tysabri. Accordingly, the methods of treating a disease associated with TREM2 loss of function (e.g., human TREM2 loss of function) described herein can further include administering a second agent to the subject in need of treatment.

The term “combination” refers to either a fixed combination in one dosage unit form, or a combined administration where a compound of the present invention and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g. a compound of the present invention and a combination partner, are both administered to a patient simultaneously in the form of a single entity or dosage. The term “non-fixed combination” means that the therapeutic agents, e.g., a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agent.

The term “pharmaceutical combination” as used herein refers to either a fixed combination in one dosage unit form, or non-fixed combination or a kit of parts for the combined administration where two or more therapeutic agents may be administered independently at the same time or separately within time intervals, especially where these time intervals allow that the combination partners show a cooperative, e.g. synergistic effect.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

Sample Preparation

Samples used in the methods described herein can be obtained from a subject using any of the methods known in the art, e.g., by biopsy or surgery. For example, a sample comprising cerebrospinal fluid can be obtained by lumbar puncture, in which a fine needle attached to a syringe is inserted into the spinal canal in the lumbar area and a vacuum is created such that cerebrospinal fluid may be sucked through the needle and collected in the syringe. CT imaging, ultrasound, or an endoscope can be used to guide this type of procedure. The sample may be flash frozen and stored at −80° C. for later use. The sample may also be fixed with a fixative, such as formaldehyde, paraformaldehyde, or acetic acid/ethanol. RNA or protein may be extracted from a fresh, frozen or fixed sample for analysis.

Pharmaceutical Compositions, Dosage, and Methods of Administration

Also provided herein are compositions, e.g., pharmaceutical compositions, for use in treatment of a TREM2-associated disease (e.g., human TREM2-associated disease). Such compositions include one or more TREM2-binding molecules (e.g., human TREM2-binding molecules) as described herein. Such compositions can further include another agent, e.g., a current standard of care for the disease to be treated.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Pharmaceutical compositions are typically formulated to be compatible with its intended route of administration. Examples of routes of administration include parenteral (e.g., intravenous, intraarterial, intraperitoneal), oral, intracranial, intrathecal, or intranasal (e.g., inhalation), intradermal, subcutaneous, or transmucosal administration. In some embodiments, the pharmaceutical compositions are formulated to deliver TREM2-binding molecules to cross the blood-brain barrier.

In some embodiments, the pharmaceutical compositions comprise one or more pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

Methods of formulating suitable pharmaceutical compositions are known in the art, see, e.g., Remington: The Science and Practice of Pharmacy. 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders, for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization.

Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Parenteral formulations can be a single bolus dose, an infusion or a loading bolus dose followed with a maintenance dose. These compositions can be administered at specific fixed or variable intervals, e.g., once a day, or on an “as needed” basis.

A suitable pharmaceutical composition for injection can comprise a buffer (e.g., acetate, phosphate or citrate buffer), a surfactant (e.g., polysorbate), optionally a stabilizer agent (e.g., human albumin), etc. Preparations for peripheral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include, e.g., water, alcoholic/aqueous solutions, emulsions or suspensions, including saline and buffered media. In some embodiments, the pharmaceutical composition comprises 0.01-0.1 M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as, for example, antimicrobials, antioxidants, chelating agents, and inert gases and the like.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798. Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the therapeutic compounds are prepared with carriers that will protect the therapeutic compounds against rapid elimination from the body, such as a controlled release formulation, including implants and microencapsulated delivery systems.

The pharmaceutical compositions can be included in a container, pack, or dispenser together with instructions for administration.

In non-limiting examples, the pharmaceutical composition containing at least one pharmaceutical agent is formulated as a liquid (e.g., a thermosetting liquid), as a component of a solid (e.g., a powder or a biodegradable biocompatible polymer (e.g., a cationic biodegradable biocompatible polymer)), or as a component of a gel (e.g., a biodegradable biocompatible polymer). In some embodiments, the at least composition containing at least one pharmaceutical agent is formulated as a gel selected from the group of an alginate gel (e.g., sodium alginate), a cellulose-based gel (e.g., carboxymethyl cellulose or carboxyethyl cellulose), or a chitosan-based gel (e.g., chitosan glycerophosphate). Additional, non-limiting examples of drug-eluting polymers that can be used to formulate any of the pharmaceutical compositions described herein include, carrageenan, carboxymethylcellulose, hydroxypropylcellulose, dextran in combination with polyvinyl alcohol, dextran in combination with polyacrylic acid, polygalacturonic acid, galacturonic polysaccharide, polysalactic acid, polyglycolic acid, tamarind gum, xanthum gum, cellulose gum, guar gum (carboxymethyl guar), pectin, polyacrylic acid, polymethacrylic acid, N-isopropylpolyacrylomide, polyoxyethylene, polyoxypropylene, pluronic acid, polylactic acid, cyclodextrin, cycloamylose, resilin, polybutadiene, N-(2-Hydroxypropyl)methacrylamide (HP MA) copolymer, maleic anhydrate-alkyl vinyl ether, polydepsipeptide, polyhydroxybutyrate, polycaprolactone, polydioxanone, polyethylene glycol, polyorganophosphazene, polyortho ester, polyvinylpyrrolidone, polylactic-co-glycolic acid (PLGA), polyanhydrides, polysilamine, poly N-vinyl caprolactam, and gellan.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Kits

Also provided herein are kits including one or more of the compositions provided herein and instructions for use. Instructions for use can include instructions for diagnosis or treatment of a TREM2-associated disease (e.g., human TREM2-associated disease). Kits as provided herein can be used in accordance with any of the methods described herein. Those skilled in the art will be aware of other suitable uses for kits provided herein, and will be able to employ the kits for such uses. Kits as provided herein can also include a mailer (e.g., a postage paid envelope or mailing pack) that can be used to return the sample for analysis, e.g., to a laboratory. The kit can include one or more containers for the sample, or the sample can be in a standard blood collection vial. The kit can also include one or more of an informed consent form, a test requisition form, and instructions on how to use the kit in a method described herein. Methods for using such kits are also included herein. One or more of the forms (e.g., the test requisition form) and the container holding the sample can be coded, for example, with a bar code for identifying the subject who provided the sample.

One skilled in the art will recognize many methods and materials similar or equivalent to those described herein, which could be used in the practice of the present invention. Indeed, the present invention is in no way limited to the methods and materials described.

### EXAMPLES

The invention is further described in the following examples, which do not limit the scope of the invention described in the claims.

### Example 1: Stabilization of Cell Surface TREM2 by SOMAmers

Materials and Methods

Reagents

Polyclonal goat anti-human TREM2 antibody (200 ug/mL) was obtained from R&D Systems, Inc. (Minneapolis, MN). Goat isotype IgG (5 mg/mL) and rabbit anti-goat IgG (Fab)′2 fragment AlexaFluor 488 (2 mg/mL) were purchased from Southern Biotech (Birmingham, Alabama). F(ab)′2 rabbit anti-goat Alexa488 was purchased from Molecular Probes (Grand Island, NY). The anti-human CD163 APC (mIgG1, 100 ug/mL), mIgG1 Alexa647 isotype control (100 ug/mL), and Streptavidin-APC (0.2 mg/mL) were purchased from Biolegend (San Diego, CA).

The cell culture medium Dulbecco's Modified Eagle Medium: Nutrient Mixture F-12 (DMEM/F12), GlutaMAX-I supplement, cell dissociation buffer, and EDTA (Ethylenediaminetetraacetic acid) were purchased from Gibco (Grand Island, NY). Bovine serum albumin (BSA), dimethyl sulfoxide (DMSO), and Greiner 384-well polypropylene V bottom plates were purchased from Sigma (St. Louis, MO). Privigen was purchased from CSL Behring (King of Prussia, PA). Phosphate-buffered saline (PBS) was purchased from Invitrogen (Grand Island, NY).

Cells

A HEK cell line stably expressing a chimeric receptor consisting of TREM2-extracellular domain and DAP12 was first described by Kleinberger et al. 2014. In analogy a TREM-2/DAP12 chimeric receptor expressing cell line was produced according to standard cell culture procedures. CHO cells were sequentially transfected with eukaryotic expression vectors for human DAP12 (hDAP12) and human TREM2 (hTREM2). Resulting CHO-hTREM2/hDAP12 double transfected cells (CHO-hTREM2) were grown in DMEM/F12 medium supplemented with 10% fetal bovine serum 0.2 mg/ml G418 and 0.2 mg/mL hygromycin. Confluent adherent CHO cells were harvested after treatment with cell dissociation buffer (Gibco 13151-014) and cell surface TREM2 was quantified by flow cytometry as described below.

Human M2a macrophages were generated from monocytes isolated from buffy coats by negative selection. M2a macrophages were generated by 5-6 days differentiation of monocytes in RPMI (supplemented 10% fetal bovine serum, and typical concentrations of Glutamax, Sodium pyruvate, Non Essential Amino Acids, HEPES) with MCSF 40 ng/mL (R&D) and 50 ng/mL IL4 (R&D). M2a macrophages were harvested with cell dissociation buffer (Gibco 13151-014) and cell surface TREM2 was assessed by flow cytometry as described below.

Detection of Cell Surface TREM2 by Flow Cytometry

Cells were stained for TREM2 surface expression in FACS buffer (PBS, containing 10% heat-inactivated human serum AB male (Invitromex), 0.05% w/v sodium azide, 5 mM EDTA, 1 mg/mL Privigen (Mg) in PBS using goat anti-human TREM2 (R&D) or an appropriate isotype control. Cells were counterstained with F(ab)′2 fragments of an rabbit anti-goat IgG polyclonal antibody conjugated with Alexa488 (LifeTechnologies). All stainings were performed on ice for 30 min Cells were assessed for TREM2 surface expression on a BD LSR Fortessa or a FacsCalibur flow cytometer. Median fluorescence intensities corresponding to the degree of anti-TREM2 bound to cells were evaluated using FlowJo (Millipore INC)

TREM2 Surface Stabilization by SOMAmers

Custom-made TREM2-specific SOMAmer libraries were generated using human TREM2-Fc Fusion protein (R&D Systems, Inc) or TREM2-HIS as “target antigen”. SOMAmers were dissolved in DMSO at 1001.1M and tested for their TREM2 specificity by binding to immobilized TREM2 fusion proteins. Exclusive binding to human TREM2 proteins was confirmed and no binding to other recombinant proteins containing immunoglobulin/IGSF domains such as human IgG1, TREM1, SIRPb1, CD47 was observed (data not shown).

Binding of SOMAmers to cellular TREM2 was assessed by incubation of biotinylated SOMAmers at various concentrations with CHO-hTREM2 cells. In this case binding of SOMAmers to cell surface TREM2 was done on ice for 20-30 min to prevent modulation of cell surface TREM2. Binding was conducted in RPMI or DMEM cell culture medium containing 10% fetal bovine serum for 30 min on ice. Cells were thereafter washed with FACS buffer and bound biotinylated SOMAmers were detected via streptavidin-APC conjugate (Biolegend, Inc). Median fluorescence intensities corresponding to the degree of TREM2 SOMAmer bound to cells were acquired on a FacsCalibur flow cytometer and evaluated using FlowJo (Millipore INC).

Statistical Analysis

All data were expressed as mean±SD Statistical analysis was performed by a Student's t Test (Excel, Microsoft, USA) or ANOVA, as indicated. The significance level was set at p<0.05.

Results

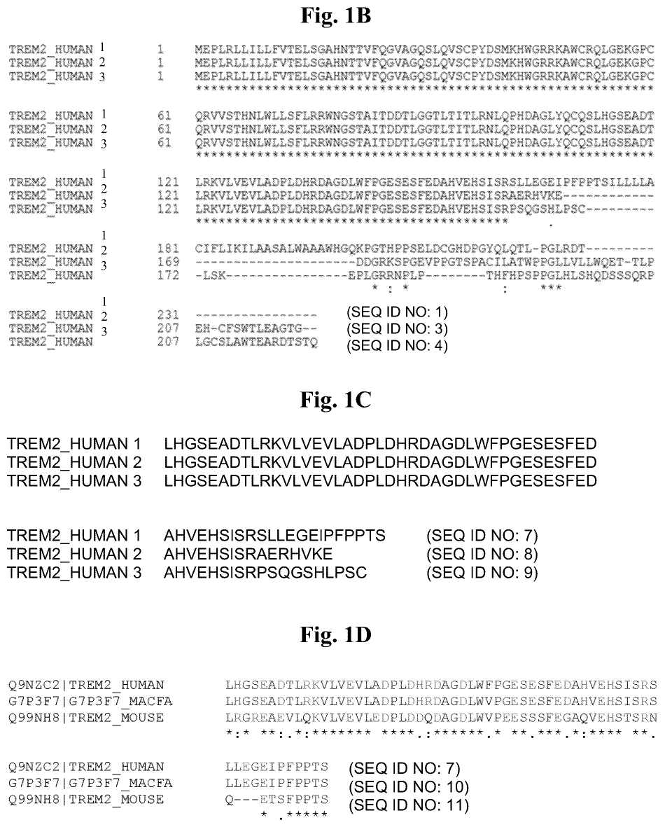

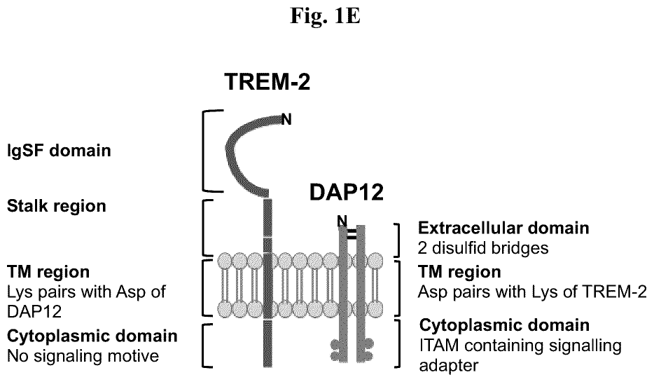

Candidate TREM2 SOMAmers were tested for binding to CHO control cells or CHO-hTREM2 cells. As shown in FIG. 2, a number of TREM2 SOMAmers were found to specifically bind cell surface human TREM2. Concentration-dependent binding of several TREM2 SOMAmers to CHO-hTREM2 cells was shown in FIG. 3. These date indicate that TREM2 SOMAmers have different binding affinities to cellular human TREM2.

TREM2 SOMAmers were tested for their ability to stabilize or increase cell surface human TREM2, e.g., by modulating homeostatic dynamics of shedding or internalization. CHO-hTREM2 cells or M2a macrophages were incubated with candidate SOMAmers at indicated concentrations in cell culture medium at 37° C. for 3 hours. Cells were then assessed for surface human TREM2 by flow cytometry. A majority of the tested SOMAmers did not modulate the cell surface human TREM2, but surprisingly, several TREM2-specific SOMAmers increased cell surface human TREM2 level. As shown in FIG. 4, TREM2 SOMAmer UX95-90BC stabilized and increased cell surface human TREM-2 on CHO-hTREM2 cells, whereas the control SOMAmer EX83-95HC did not bind to cell surface human TREM2. Another TREM2 SOMAmer KX86-94WC also stabilized cell surface human TREM2 on CHO-hTREM2 cells (FIG. 5A); and more importantly, KX86-94WC also stabilized cell surface human TREM2 on physiologically relevant primary human M2a macrophages (FIG. 5B). The core sequences of the TREM2 SOMAmers and a control SOMAmer were listed in Table 1 below.

TABLE 1
Sequence of Selected TREM2 SOMAmers

| SOMAmer | | |
|---|---|---|
| ID | Core Sequence | SEQ ID NO |
| UX85-90BC | CCCTCGAPCCGGPGGCGGAPCCPAAGPAPPGGCCCGG | 12 |
| | PCCGCCAAGAAGA | |
| KX86-94WC | CCCTCPGPCAACCPCCACACAGGCCPPPGAPGCAAGP | 13 |
| | APACAGPGGAAGA | |
| EX83-95HC | CCCTCCCPAPCGCAAPGACACAPPGGGGPCPCGAGGP | 14 |
| (control) | GCGCGAPCGAAGA | |

Note:
“P” refers to the NapdU modified dUTP residue.

Taken together, these data indicate that SOMAmers that direct bind to the extracellular domain of human TREM2 can stabilize cell surface human TREM2 and increase the cell surface expression of human TREM2 over time.

### Example 2: Identification of Key Proteases Responsible for TREM2 Shedding

Materials and Methods

THP-1 cells, an established human monocytic cell line, were cultured in RPMI with 10% FCS, 1% PS, 1% NaPy, and used for the assays. The protease inhibitors (PI) used in the assay were developed internally, including a broad ADAM inhibitor (PI1), a TACE specific inhibitor (PI2), a γ-secretase inhibitor (PI3), and a metallo-protease inhibitor (PI4, with some TACE inhibitory activity at high doses).

The cell surface TREM2 was detected with FACS assays as described above. Soluble/shed TREM2 was detected by Delfia assay. Briefly, capture antibody (1 lag/ml, goat polyclonal anti-human TREM2; R&D Systems, AF1828) was coated at 20 μl/well in 384 Maxisorp (Nunc 464718) wells overnight at room temperature (RT). Antibody solution was flicked of and PBS with 1% BSA and 0.05% Tween was added and incubated for 1 hour at RT. Thereafter, three washes with PBS with 1% BSA and 0.05% Tween were applied. For assessment of standard or sample, 20 μl/well were used and incubated for 2 hours at RT on a plate shaker. This was followed by 3 washes with PBS with 1% BSA and 0.05% Tween. 20 μl of the detection antibody (goat anti-TREM-biotin, 500 ng/ml; R&D Systems BAF1828) were added to each well and incubated for 2h at RT. Thereafter, three washes with PBS with 1% BSA and 0.05% Tween were applied. Detection with Streptavidin-europium complex (diluted 1/4000, Perkin Elmer 1244-360) was run with 20 μl/well and incubated for 45 min at RT on a plate shaker. This was followed by three washes with PBS with 1% BSA and 0.05% Tween. Thereafter, 20 μl Delfia enhancement solution (Perkin Elmer 1244-104) were added and 15 min later plates were assessed in a Victor 2 instrument (Perkin Elmer).

Results

The expression of human TREM2 at the cell surface of THP-1 cells was increased by both a TACE specific inhibitor (PI2) and an ADAM inhibitor (PI1), which inhibits both TACE (ADAM17) and ADAM10, in a dose-dependent manner with an IC5 0 of 0.3 μM for both compounds (FIG. 6A). Inhibitors of other classes of proteases, such as an inhibitor of γ-secretases (PI3) or an inhibitor of metallo-proteases (PI4, with some TACE inhibitory activity at high doses) were not efficacious in stabilizing human TREM2 cell surface expression.

Consistent with the above finding, the production of soluble TREM2 fragments from THP-1 cells was reduced after administration of both 1 μM of the TACE specific inhibitor PI2 and the broad ADAM inhibitor PI1 (FIG. 6B). The γ-secretase inhibitor PI3 and the metallo-protease inhibitor PI4, when used at the same concentration, did not reduce shedding of human TREM2.

In THP-1 cells, shedding of TREM2 was accelerated by treatment with 50 ng/ml phorbol-myristate-acid (PMA) for 20 minutes (FIG. 6C). In the absence of PMA, TREM2-cell surface expression was stabilized by both a TACE specific inhibitor (PI2) and broad ADAM inhibitor (PI1). Treatment with PMA strongly reduced human TREM2 cell surface expression to non-detectable levels. Human TREM2 cell surface expression was rescued with concomitant treatment with either the TACE specific inhibitor PI2 or the broad ADAM inhibitor PI1.

In summary, FIGS. 6A-C show that TREM2 was constitutively shed in human monocytic cell line THP-1 cells. Shedding of human TREM2 was enhanced after brief treatment of these cells with PMA. Constitutive and induced shedding of human TREM2 was inhibited by both a TACE specific inhibitor and a broad ADAM inhibitor, indicating that TACE and ADAM10 contribute to human TREM2 shedding.

### Example 3: TACE and ADAM10 Cleave TREM2 in a Stalk Region Proximal to the Plasma Membrane

Materials and Methods

To map the sheddase cleavage region in human TREM2, ten constructs with TREM2 deletions and four constructs with amino acid substitution mutations of TREM2 were generated Amino acid residue numbering was given according to UniProt Q9NZC1, where the stalk region of human TREM2 corresponds to amino acid residues 113-174 of SEQ ID NO: 1. The deletion or substitution mutations were introduced using the QuickChange site-directed mutagenesis kit (Agilent) using primers that carried the desired mutations.

The following six truncation mutants and four deletion mutants were generated:
trunc 1: δ169-174,
trunc 2: δ164-174,
trunc 3: δ159-174,
trunc 4: δ154-174,
trunc 5: δ144-174,
trunc 6: δ134-174,
del 1: δ167-172,
del 2: δ164-169,
del 3: δ159-164,
del 4: δ149-158.

The following four substitution mutations were made:

E1:
EHSISRSLL (SEQ ID NO: 15) → YGGWGGWP (SEQ ID
NO: 16);
E2:
PFPP (SEQ ID NO: 17) → AFAA (SEQ ID NO: 18),
E3:
PFPP (SEQ ID NO: 17) → WFPW (SEQ ID NO: 19),
E4:
E1 + E3.

Individual deletion mutants were transfected together with human DAP12 cDNA into HEK-FT cells. 48 hours later, cell surface expression of human TREM2 was assessed in the presence or absence of PMA treatment for 30 min. The ratio of the cell surface expression of human TREM2 in both conditions was calculated.

Results

The membrane-proximal deletions (trunc1: δ169-174, trunc3: δ159-174) led to a considerable reduction of shedding, indicating that amino acids which are important for shedding are located in this region (FIG. 7). A second set of mutations showed that there might be a second region involved in shedding of TREM2 ectodomain, since deletion of amino acid residues 159-164 (del3) stabilized human TREM2 at the cell surface (FIG. 7).

In a next step, these two regions were investigated in greater detail with substitution mutants. Amino acid residues were replaced with amino acids that were not seen in canonical ADAM10 or TACE cleavage sites. Replacing amino acids 169-172 with bigger, more hydrophobic residues (E3) reduced the no-PMA/PMA shedding ratio (FIG. 7). This effect was even more pronounced with substitution mutant E1 (amino acids 156-164, (FIG. 7). The E4 mutant, which combines both E1 and E3 mutations, showed the most consistent preservation of human TREM2 cell surface expression (FIG. 7).

In summary, the deletion and mutation analysis revealed two sites within the human TREM2 stalk region proximal to the plasma membrane as possible cleavage or recognition sites for ADAM10 and TACE.

### Example 4: Identification of TACE Cleavage Sites in TREM2

Material and Methods

The cleavage sites of human TACE were assessed by high performance liquid chromatography-high resolution mass spectrometry (HPLC-HRMS). Human TACE (ADAM-17) recombinant catalytic domain was expressed in house in bacculovirus and stored in aliquots at −20° C.

Human TREM2 stalk region peptides were synthesized by Biosyntan GmbH, Berlin Germany, using standard solid phase methodology and N-terminally labeled with 7-Methoxycoumarinyl-3-acetyl (Mca). Peptides were dissolved in dimethylsulfoxide (DMSO) at 10 mM, diluted with DMSO to 2 mM, and further diluted in 10 mM 2-(4-(2-Hydroxyethyl)-1-piperazinyl)-ethansulfonic acid (HEPES), pH 7.5 to the desired stock solution concentration. The overlapping peptides cover the entire stalk region of human TREM2, with the following peptide sequences:

Peptide 76:
(SEQ ID NO: 20)
SLHGSEADTLRKVLVEVLAD
Peptide 79:
(SEQ ID NO: 21)
VEVLADPLDHRD
Peptide 77:
(SEQ ID NO: 22)
PLDHRDAGDLWFPGESESFE
Peptide 80:
(SEQ ID NO: 23)
ESESFEDAHVEH
Peptide 78:
(SEQ ID NO: 24)
DAHVEHSISRSLLEGEIPFP.

The cleavage reaction was initiated by mixing Mca-labeled peptide (final concentration 5 μM), TACE, and 10 mM HEPES buffer (pH 7.5) in an eppendorf tube, which was kept at room temperature. Aliquotes were withdrawn and injected in a Thermo Systems Products HPLC instrument with F3000 fluorescence detector. Separation was accomplished by gradient elution at a Nucleosil 150×3 mm 100-3 C18 column (Machery Nagel, Oensingen, Switzerland) using water/0.1% trifluoroacetic acid as solvent A and water/acetonitrile/trifluoroacetic acid 30/70/0.05% as solvent B. Detection wavelengths were set to 324 nm (excitation) and 400 nm (emission). Fifty microliter from the cleavage assay mixture were injected at various time points, from 4 hours to 5 days, and a clear separation of parent peptide and cleavage products was observed.

Results

Using HPLC/HRMS, no TACE cleavages were observed for Peptides 76, 79, 77 and 80. For Peptide 78, two main cleavage products by TACE were found: DAHVEH (termed X01, SEQ ID NO: 25) and SISRSLLEGEIPFP (termed X02, SEQ ID NO: 26), as shown in FIG. 8. A minor cleavage product X03 (SRSLLEGEIPFP, SEQ ID NO: 27) was also observed (FIG. 8), however, the counterpart of X03 was not identified.

These data indicate that human TACE cleaves the stalk region of human TREM2 isoform 1 between Histidine 157 and Serine 158 of SEQ ID NO: 1 (FIG. 8, amino acid numbering is given according to UniProt Q9NZC1). Histidine 157 and Serine 158 are conserved in human TREM2 isoforms 2 and 3 (FIGS. 1B and 1C), suggesting TACE cleaves these human TREM2 isoforms at the same site.

### Example 5: Stabilization of Cell Surface TREM2 by an Anti-TREM2 Antibody

Material and Methods

A cell line expressing human TREM2 and human DAP12 was produced according to standard cell culture procedures. CHO cells were sequentially transfected with eukaryotic expression vectors for human DAP12 and human TREM2. Resulting CHO-hDAP12-hTREM2 double transfected cells were grown in DMEM/F12 medium supplemented with 10% fetal bovine serum, 0.1 mg/ml G418, and 0.5 mg/ml hygromycin. Confluent adherent CHO cells were harvested using cell dissociation buffer (Gibco 13151-014) and cell surface TREM2 was quantified by flow cytometry as described below.

The CHO-hDAP12-hTREM2stalk-hTREM1-IGSF cell line was generated by sequentially transfecting CHO cells with eukaryotic expression vectors for human DAP12 and a chimeric cDNA construct where the IGSF domain of hTREM2 cDNA was replaced by the IGSF domain of hTREM1 cDNA. Resulting CHO-hDAP12-hTREM2stalk-hTREM1-IGSF double transfected cells were grown in DMEM/F12 medium supplemented with 10% fetal bovine serum 0.1 mg/ml G418 and 0.5 mg/ml hygromycin. A clone that showed similar DAP12 and hTREM2stalk-hTREM1-IGSF expression levels as the corresponding wild type (WT) TREM2 cell line described above was chosen for all further experimentations. Confluent adherent CHO cells were harvested using cell dissociation buffer (Gibco 13151-014) and cell surface expressed chimeric and hTREM2stalk-hTREM1-IGSF was quantified by flow cytometry as described below.

TREM2 cell surface stabilization experiment: CHO-hDAP12-hTREM2 cells or CHO-hDAP12-hTREM2stalk-hTREM1-IGSF cells were dissociated from cell culture flasks and 30,000 cells were incubated with 20 nM of either a goat anti-human TREM2 antibody AF1828 (polyclonal antibody raised against recombinant human TREM2 His19-Ser174, R&D systems) or biotinylated AF1828 (BAF1828, R&D systems), or an isotype control AF108 (R&D systems) or biotinylated AF108 (BAF108, R&D systems), for 30 min at RT, followed by PMA treatment (50 ng/ml, Sigma Aldrich, P8139) for another 30 min at 37° C. Cells were placed on ice and processed for FACS staining.

TREM2 cell surface stabilization experiment under conditional shedding conditions: CHO-hDAP12-hTREM2 were plated on ultra-low adherence plates and incubated with different antibodies for indicated time points at RT. Cells were placed on ice and processed for FACS staining.

Cells were stained for TREM2 surface expression in FACS buffer (PBS, containing 10% heat-inactivated human serum AB male (Invitromex), 0.05% w/v sodium azide, 5 mM EDTA, 1 mg/mL Privigen (Mg) in PBS using goat anti-human TREM2 BAF1828. Cells were counterstained with Streptavidin, Alexa Fluor® 488 Conjugate (Molecular Probes). All stainings were performed on ice for 30 min. Cells were assessed for TREM2 surface expression on a BD LSR Fortessa or a FacsCalibur or BD FACS Canto II flow cytometer. Median fluorescence intensities corresponding to the degree of anti-TREM2 bound to cells were evaluated using FlowJo (Millipore INC).

All data were expressed as mean±SD. Statistical analysis was performed by a Student's t Test (Prism software, USA). The significance level was set at p<0.05.

Results

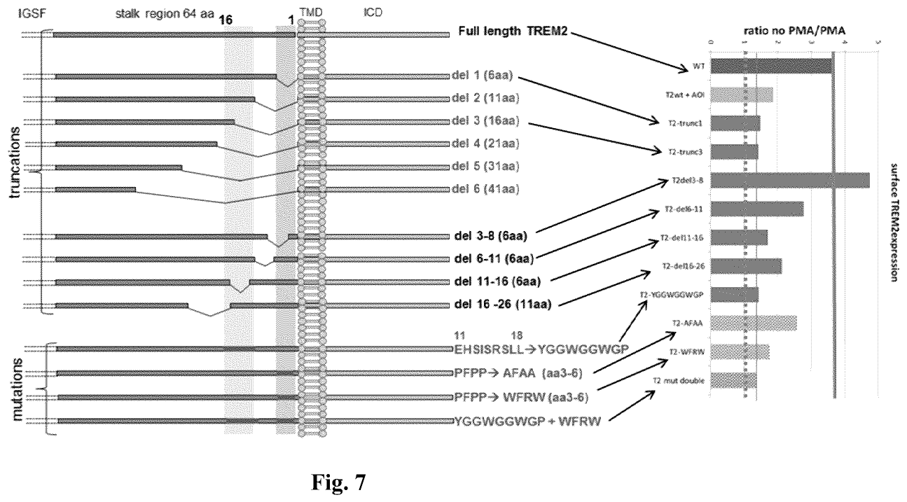

The biotinylated goat anti-human TREM2 antibody BAF1828 and the biotinylated goat isotype control BAF108 were tested for stabilization of cell surface hTREM2 recombinantly expressed on CHO-hDAP12-hTREM2 cells with or without PMA challenge. As shown in FIG. 9, pre-treatment with BAF1828 but not with the isotype control (BAF108, p<0.05) preserved human TREM2 cell surface expression in the presence of PMA indicating that shedding of human TREM2 is reduced after BAF1828 binding.

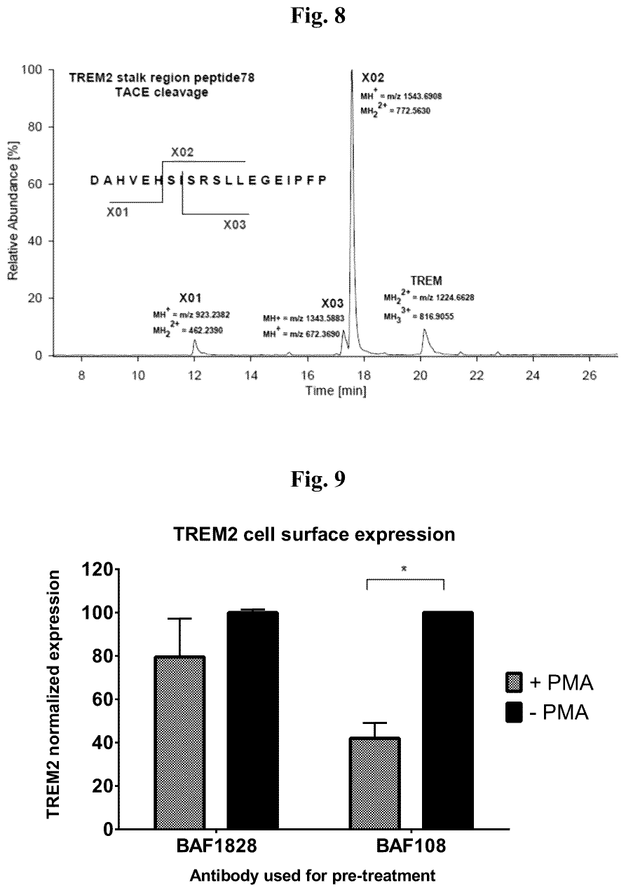

To assess stabilization of human TREM2 towards constitutive shedding, CHO-hDAP12-hTREM2 cells were plated on ultra-low adherence plates and treated for 16 hours with 25 nM of BAF1828, BAF108, AF1828, or a corresponding goat IgG isotype control, or with 5 μM of PI1 (a ADAM10 and TACE inhibitor) or cell culture medium (FIG. 10). The anti-TREM2 antibodies, BAF1828 and AF1828, as well as the ADAM and TACE inhibitor PI1 increased cell surface expression of human TREM2, indicating reduction of ectodoamain cleavage (FIG. 10).

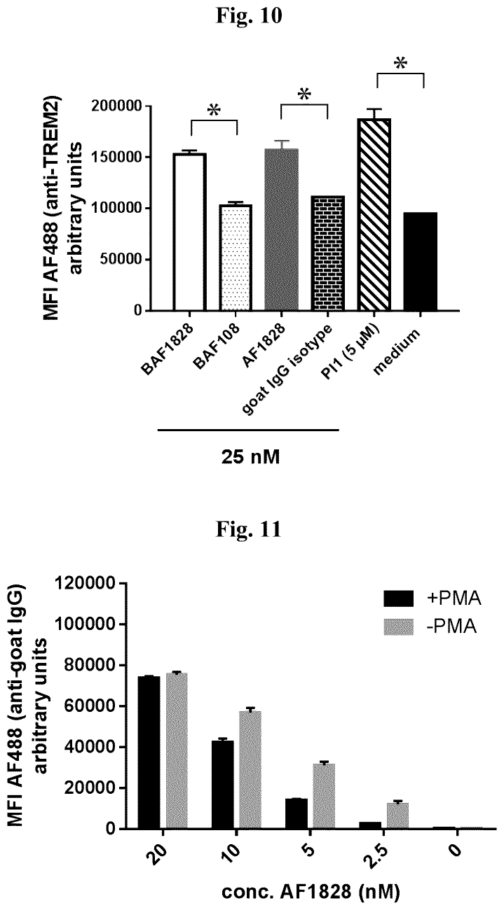

Next, we investigated in CHO-hDAP12-hTREM2 cells the dose dependence of human TREM2 cell surface stabilization by AF1828. To this end we used the PMA induced shedding protocol and incubated the cells with 2.5, 5, 10, or 20 nM of AF1828 for 30 minutes before PMA treatment (FIG. 11). Increasing concentrations of the antibody lead to increased protection against PMA-induced shedding, and at the concentration of 20 nM of AF1828, full protection was observed (FIG. 11).

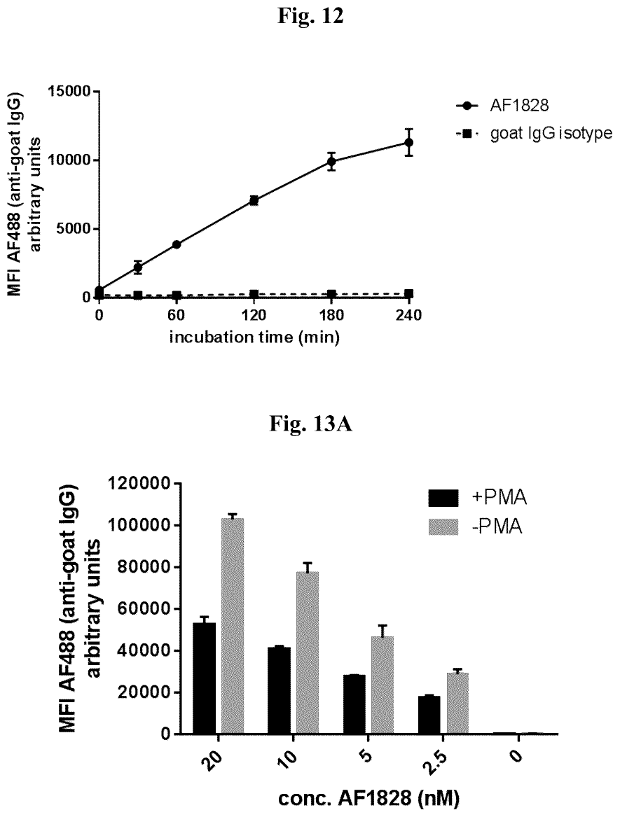

To elucidate duration dependency of AF1828 pre-incubation on human TREM2 cell surface stabilization, we used the constitutive shedding protocol and performed different pre-treatment periods up to 6 hours with 25 nM of AF1828 or the goat isotype control (FIG. 12). FACS staining with anti-goat AF488 labelled antibody revealed an accumulation of human TREM2 over time at the cell surface only in the AF1828 pre-incubated cells, but not in isotype control treated cells (FIG. 12). These data indicate that newly synthesized human TREM2 molecules arriving at the cell surface are protected against shedding in the presence of AF1828 (FIG. 12).

In summary, these data demonstrate a concentration and duration dependent stabilization of cell surface human TREM2 by AF1828 in CHO-hDAP12-hTREM2 cells; and both PMA-induced shedding as well as constitutive shedding of human TREM2 is reduced by AF1828 treatment.

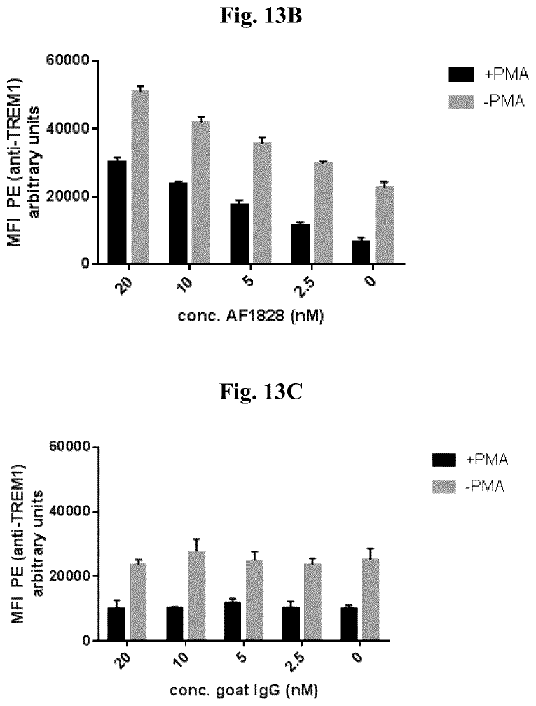

To understand which protein domains of human TREM2 are critical for AF1828 mediated stabilization, we established a “switch-construct” cell line that expressed a human TREM2/human TREM1 chimeric protein together with DAP12 (see Material and Methods). Employing the PMA-induced shedding protocol, the CHO-hDAP12-hTREM2stalk-hTREM1-IGSF cells were incubated with 2.5, 5, 10, or 20 nM of AF1828 for 30 min before PMA treatment (FIG. 13A). Using FACS staining with anti-goat AF488, we found that increasing concentrations of AF1828 lead to increased protection against PMA-induced shedding, but even at a concentration of 20 nM of AF1828, full protection was not achieved (FIG. 13A). This is in contrast to the findings in the CHO-hDAP12-hTREM2 cell line, where full protection was achieved at 20 nM of AF1828 (see FIG. 11). These results indicate that human TREM2 stalk region binders within the AF1828 polyclonal antibody confer shedding protection, since in the switch construct cell line that contains only the human TREM2 stalk region, but not the human TREM2 IGSF domain, cell surface stabilization is achieved. Furthermore, some human TREM2 IGSF region binders might also contribute to shedding protection since the extent of shedding protection in the switch construct cell line is reduced compared to the full-length WT human TREM2 cell line (compare FIGS. 11 and 13A).

To corroborate these findings, we used the same AF1828-preincubated and PMA-treated cells, but carried out a FACS staining independent of the antibody used for stabilization. To this end, direct TREM1 staining against the human TREM1 IGSF domain of the chimeric construct was performed using an anti-TREM1-PE antibody (R&D systems) for FACS (FIG. 13B). The results are very similar to the findings presented in FIG. 13A. Finally, we carried out a control experiment where different concentrations of a goat isotope control antibody were used for stabilization instead of AF1828 (FIG. 13C) Staining with the anti-TREM1-PE antibody revealed that the isotype control antibody has no effect on human TREM2 surface stabilization at all tested concentrations (FIG. 13C), which substantiates the specificity of AF1828 on human TREM2 cell surface stabilization. In summary, these data demonstrate that within the AF1828 polyclonal antibody, both human TREM2 stalk region binders as well as human TREM2 IGSF region binders contribute to protection against sheddase cleavage. Furthermore, human TREM2 cell surface stabilization by AF1828 is specific, since an isotype control did not confer such protection.

These data indicate that an anti-human TREM2 antibody can stabilize cell surface human TREM2.

Unless defined otherwise, the technical and scientific terms used herein have the same meaning as they usually understood by a specialist familiar with the field to which the disclosure belongs.

Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein. Unless indicated otherwise, each of the references cited herein is incorporated in its entirety by reference.

Claims to the invention are non-limiting and are provided below.

Although particular aspects and claims have been disclosed herein in detail, this has been done by way of example for purposes of illustration only, and is not intended to be limiting with respect to the scope of the appended claims, or the scope of subject matter of claims of any corresponding future application. In particular, it is contemplated by the inventors that various substitutions, alterations, and modifications may be made to the disclosure without departing from the spirit and scope of the disclosure as defined by the claims. The choice of nucleic acid starting material, clone of interest, or library type is believed to be a matter of routine for a person of ordinary skill in the art with knowledge of the aspects described herein. Other aspects, advantages, and modifications considered to be within the scope of the following claims. Those skilled in the art will recognize or be able to ascertain, using no more than routine experimentation, many equivalents of the specific aspects of the invention described herein. Such equivalents are intended to be encompassed by the following claims. Redrafting of claim scope in later filed corresponding applications may be due to limitations by the patent laws of various countries and should not be interpreted as giving up subject matter of the claims.

1. A molecule that specifically binds to a human TREM2 protein on the cell surface of a TREM2-expressing cell, wherein the molecule is not an antibody or an antigen binding fragment thereof.

2. The molecule of claim 1, wherein the molecule is selected from the group consisting of a low molecular weight compound and an aptamer.

3. The molecule of claim 1, wherein the molecule specifically binds to an extracellular domain of the human TREM2 protein.

4. The molecule of claim 3, wherein the extracellular domain of the human TREM2 protein comprises the amino acid residues 14 to 174 of SEQ ID NO: 1, the amino acid residues 14 to 168 of SEQ ID NO: 3, or the amino acid residues 14 to 171 of SEQ ID NO: 4.

5. The molecule of claim 1, wherein the molecule specifically binds to a stalk region of the human TREM2 protein.

6. The molecule of claim 5, wherein the stalk region of the human TREM2 protein comprises an amino acid sequence of any one of SEQ ID NO: 7, 8, or 9.

7. The molecule of claim 1, wherein the cell is selected from a macrophage, dendritic cell, osteoclast, microglia, lung epithelial cell, or hepatocarcinoma cell.

8. The molecule of claim 1, wherein the molecule stabilizes the human TREM2 protein and/or reduces shedding of the ectodomain of the human TREM2 protein.

9. The molecule of claim 8, wherein the molecule stabilizes the human TREM2 protein on the cell surface of a TREM2-expressing cell selected from a macrophage, dendritic cell, osteoclast, microglia, lung epithelial cell, or hepatocarcinoma cell.

10. The molecule of claim 2, wherein the aptamer is an oligonucleotide aptamer or a peptide aptamer.

11. The molecule of claim 2, wherein the aptamer is a slow off-rate modified aptamer (SOMAmer).

12. The molecule of claim 11, wherein the SOMAmer comprises a sequence of SEQ ID NO: 12 or 13.

13. A pharmaceutical composition comprising the molecule of claim 1 and a pharmaceutically acceptable carrier.

14. A method of treating a disease associated with human TREM2 loss of function in a subject in need thereof, the method comprising administering to the subject the pharmaceutical composition of claim 13.

15. A method of treating a disease associated with human TREM2 loss of function in a subject in need thereof, the method comprising administering to the subject a therapeutically effective amount of the molecule of claim 1.

16. The method of claim 15, wherein the molecule is selected from the group consisting of a low molecular weight compound and an aptamer.

17. The method of claim 15, wherein the molecule specifically binds to an extracellular domain of the human TREM2 protein.

18. The method of claim 17, wherein the extracellular domain of the human TREM2 protein comprises the amino acid residues 14 to 174 of SEQ ID NO: 1, the amino acid residues 14 to 168 of SEQ ID NO: 3, or the amino acid residues 14 to 171 of SEQ ID NO: 4.

19. The method of claim 15, wherein the molecule specifically binds to a stalk region of the human TREM2 protein.

20. The method of claim 19, wherein the stalk region of the human TREM2 protein comprises an amino acid sequence of any one of SEQ ID NO: 7, 8, or 9.

21. The method of claim 15, wherein the method comprises:
assaying a cell surface human TREM2 level in a sample obtained from a subject;
selecting a subject whose cell surface human TREM2 level is lower than a reference level, wherein the reference level is the cell surface human TREM2 level in a sample obtained from a healthy subject; and
administering to the selected subject the therapeutically effective amount of the molecule.

22. The method of claim 21, wherein the sample comprises cerebrospinal fluid.

23. The method of claim 21, wherein the cell surface human TREM2 level in a sample is determined by an assay selected from flow cytometry, immunohistochemistry, Western blotting, immunofluorescent assay, radioimmunoassay (RIA), enzyme-linked immunosorbent assay (ELISA), homogeneous time resolved fluorescence (HTRF), or positron emission tomography (PET).

24. The method of claim 15, wherein the disease associated with human TREM2 loss of function is a neuroinflammatory or neurodegenerative disease selected from Alzheimer's disease, frontotemporal dementia, Parkinson's disease, amyotrophic lateral sclerosis, Nasu-Hakola disease, multiple sclerosis, amyotrophic lateral sclerosis (ALS), antiNMDA receptor encephalitis, autism, brain lupus (NP-SLE), chemo-induced peripheral neuropathy (CIPN), postherapeutic neuralgia, chronic inflammatory demyelinating polyneuropathy (CIDP), epilepsy, Guillain-Barre Syndrome (GBS), inclusion body myositis, lysosomal storage diseases, sphingomyelin lipidose (Niemann-Pick C), mucopolysaccharidose II/11IB, metachromatic leukodystrophy, multifocal motor neuropathy, Myasthenia Gravis, Neuro-Behcet's Disease, neuromyelitis optica (NMO), optic neuritis, polymyositis, dermatomyositis, Rasmussen's encephalitis, Rett's Syndrome, stroke, transverse myelitis, traumatic brain injury, spinal cord injury, viral encephalitis, or bacterial meningitis.

25. The method of claim 15, wherein the molecule stabilizes the human TREM2 protein and/or reduces shedding of the ectodomain of the human TREM2 protein.

26. The method of claim 25, wherein the molecule stabilizes the human TREM2 protein on the cell surface of a TREM2-expressing cell selected from a macrophage, dendritic cell, osteoclast, microglia, lung epithelial cell, or hepatocarcinoma cell.

27. The method of claim 16, wherein the aptamer is an oligonucleotide aptamer or a peptide aptamer.

28. The method of claim 16, wherein the aptamer is a slow off-rate modified aptamer (SOMAmer).

29. The method of claim 28, wherein the SOMAmer comprises a sequence of SEQ ID NO: 12 or 13.

30. The method of claim 15, wherein the molecule is administered to the subject through an oral, intravenous, intracranial, intrathecal, subcutaneous, or intranasal route.

31. The method of claim 15, the method further comprising administering a second agent to the subject.
